(19) United States
(12) Patent Application Publication
(10) Pub. No.: US 2020/0054633 A1
(43) Pub. Date: Feb. 20, 2020

(54) COMBINATION THERAPIES FOR TREATING INFLUENZA VIRUS INFECTION

(71) Applicant: Vertex Pharmaceuticals Incorporated, Boston, MA (US)

(72) Inventors: Robert S. Kauffman, Brookline, MA (US); Sarah Marie Robertson, Somerville, MA (US)

(21) Appl. No.: 16/597,326

(22) Filed: Oct. 9, 2019

Related U.S. Application Data
(63) Continuation of application No. PCT/US2018/027264, filed on Apr. 12, 2018.
(60) Provisional application No. 62/593,356, filed on Dec. 1, 2017.
(60) Provisional application No. 62/484,563, filed on Apr. 12, 2017.

(51) Int. Cl. A61K 31/506; A61K 31/196; A61P 31/16

(52) U.S. Cl. CPC A61K 31/506; A61P 31/16; A61K 31/196

(57) ABSTRACT

The present invention relates to mono and combination therapies that are useful for inhibiting influenza virus replication, treating or reducing the severity of influenza infections in patients, and prophylactically preventing or reducing the incidence of influenza infections in patients. The combinations described herein include a compound having the structure

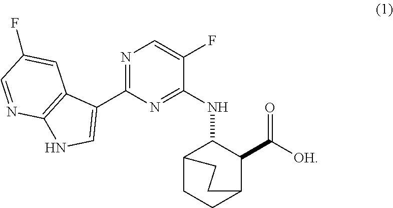 [SMILES: O=C(O)[C@H]1C2CCC(CC2)[C@@H]1Nc1nc(-c2c[nH]c3ncc(F)cc23)ncc1F]

### CROSS REFERENCE TO RELATED APPLICATION

This PCT application claims the benefit of U.S. provisional application no. 62/484,563, filed on Apr. 12, 2017 and U.S. provisional application No. 62/593,356, filed on Dec. 1, 2017. Each of these documents is hereby incorporated by reference in its entirety.

### FIELD OF THE INVENTION

The present invention relates to mono and combination therapies that are useful for inhibiting influenza virus replication, treating or reducing the severity of influenza infections in patients, and prophylactically preventing or reducing the incidence of influenza infections in patients.

### BACKGROUND

Influenza spreads around the world in seasonal epidemics, resulting in the deaths of hundreds of thousands annually—millions in pandemic years. For example, three influenza pandemics occurred in the 20th century and killed tens of millions of people, with each of these pandemics being caused by the appearance of a new strain of the virus in humans. Often, these new strains result from the spread of an existing influenza virus to humans from other animal species.

Influenza is primarily transmitted from person to person via large virus-laden droplets that are generated when infected persons cough or sneeze; these large droplets can then settle on the mucosal surfaces of the upper respiratory tracts of susceptible individuals who are near (e.g. within about 6 feet) infected persons. Transmission might also occur through direct contact or indirect contact with respiratory secretions, such as touching surfaces contaminated with influenza virus and then touching the eyes, nose or mouth. Adults might be able to spread influenza to others from 1 day before getting symptoms to approximately 5 days after symptoms start. Young children and persons with weakened immune systems might be infectious for 10 or more days after onset of symptoms.

Influenza viruses are RNA viruses of the family Orthomyxoviridae, which comprises five genera: Influenza virus A, Influenza virus B, Influenza virus C, ISA virus and Thogoto virus.

The Influenza virus A genus has one species, influenza A virus. Wild aquatic birds are the natural hosts for a large variety of influenza A. Occasionally, viruses are transmitted to other species and may then cause devastating outbreaks in domestic poultry or give rise to human influenza pandemics. The type A viruses are the most virulent human pathogens among the three influenza types and cause the most severe disease. The influenza A virus can be subdivided into different serotypes based on the antibody response to these viruses. The serotypes that have been confirmed in humans, ordered by the number of known human pandemic deaths, are: H1N1 (which caused Spanish influenza in 1918), H2N2 (which caused Asian Influenza in 1957), H3N2 (which caused Hong Kong Flu in 1968), H5N1 (a pandemic threat in the 2007-08 influenza season), H7N7 (which has unusual zoonotic potential), H1N2 (endemic in humans and pigs), H9N2, H7N2 , H7N3 and H1ON7.

The Influenza virus B genus has one species, influenza B virus. Influenza B almost exclusively infects humans and is less common than influenza A. The only other animal known to be susceptible to influenza B infection is the seal. This type of influenza mutates at a rate 2-3 times slower than type A and consequently is less genetically diverse, with only one influenza B serotype. As a result of this lack of antigenic diversity, a degree of immunity to influenza B is usually acquired at an early age. However, influenza B mutates enough that lasting immunity is not possible. This reduced rate of antigenic change, combined with its limited host range (inhibiting cross species antigenic shift), ensures that pandemics of influenza B do not occur.

The Influenza virus C genus has one species, influenza C virus, which infects humans and pigs and can cause severe illness and local epidemics. However, influenza C is less common than the other types and usually seems to cause mild disease in children.

Influenza A, B and C viruses are very similar in structure. The virus particle is 80-120 nanometers in diameter and usually roughly spherical, although filamentous forms can occur. Unusual for a virus, its genome is not a single piece of nucleic acid; instead, it contains seven or eight pieces of segmented negative-sense RNA. The Influenza A genome encodes 11 proteins: hemagglutinin (HA), neuraminidase (NA), nucleoprotein (NP), M1, M2, NS1, NS2(NEP), PA, PB1, PB1-F2 and PB2.

HA and NA are large glycoproteins on the outside of the viral particles. HA is a lectin that mediates binding of the virus to target cells and entry of the viral genome into the target cell, while NA is involved in the release of progeny virus from infected cells, by cleaving sugars that bind the mature viral particles. Thus, these proteins have been targets for antiviral drugs. Furthermore, they are antigens to which antibodies can be raised. Influenza A viruses are classified into subtypes based on antibody responses to HA and NA, forming the basis of the H and N distinctions (vide supra) in, for example, H5N1.

Influenza produces direct costs due to lost productivity and associated medical treatment, as well as indirect costs of preventative measures. In the United States, influenza is responsible for a total cost of over $10 billion per year, while it has been estimated that a future pandemic could cause hundreds of billions of dollars in direct and indirect costs. Preventative costs are also high. Governments worldwide have spent billions of U.S. dollars preparing and planning for a potential H5N1 avian influenza pandemic, with costs associated with purchasing drugs and vaccines as well as developing disaster drills and strategies for improved border controls.

Current treatment options for influenza include vaccination, and chemotherapy or chemoprophylaxis with anti-viral medications. Vaccination against influenza with an influenza vaccine is often recommended for high-risk groups, such as children and the elderly, or in people that have asthma, diabetes, or heart disease. However, it is possible to get vaccinated and still get influenza. The vaccine is reformulated each season for a few specific influenza strains but cannot possibly include all the strains actively infecting people in the world for that season. It may take six months for the manufacturers to formulate and produce the millions of doses required to deal with the seasonal epidemics; occasionally, a new or overlooked strain becomes prominent during that time and infects people although they have been vaccinated (as by the H3N2 Fujian flu in the 2003-2004 influenza season). It is also possible to get infected just before vaccination and get sick with the very strain that the vaccine is supposed to prevent, as the vaccine may require several weeks to become effective.

Further, the effectiveness of these influenza vaccines is variable. Due to the high mutation rate of the virus, a particular influenza vaccine usually confers protection for no more than a few years. A vaccine formulated for one year may be ineffective in the following year, since the influenza virus changes rapidly over time, and different strains become dominant.

Also, because of the absence of RNA proofreading enzymes, the RNA-dependent RNA polymerase of influenza vRNA makes a single nucleotide insertion error roughly every 10 thousand nucleotides, which is the approximate length of the influenza vRNA. Hence, nearly every newly-manufactured influenza virus is a mutant-antigenic drift. The separation of the genome into eight separate segments of vRNA allows mixing or reassortment of vRNAs if more than one viral line has infected a single cell. The resulting rapid change in viral genetics produces antigenic shifts and allows the virus to infect new host species and quickly overcome protective immunity.

Antiviral drugs can also be used to treat influenza, with neuraminidase inhibitors being particularly effective, but viruses can develop resistance to the standard antiviral drugs. Such agents can be combined with other antiviral drugs to improve influenza prophylaxis, to reduce patient recovery time for influenza infection, and to reduce the severity of influenza virus infection symptoms.

Pimodivir (Compound (1)) is a first in class PB2 subunit inhibitor of the influenza A polymerase being developed for the treatment of patients at risk of influenza-related complications, including hospitalized patients. Herein is established a safety database to determine the dose of pimodivir for further development, and the benefits of combination with oseltamivir vs monotherapy treatment.

### SUMMARY OF THE INVENTION

The present invention generally relates to therapeutic combinations comprising Compound (1) or a pharmaceutically acceptable salt thereof, and a neuraminidase inhibitor (e.g., oseltamivir or zanamivir).

The invention also generally relates to a method of treating or reducing the severity of influenza virus infection comprising administering to a patient infected with influenza from about 200 mg to about 800 mg at least once per day Compound (1) or a pharmaceutically acceptable salt thereof, wherein Compound (1) has the structure:

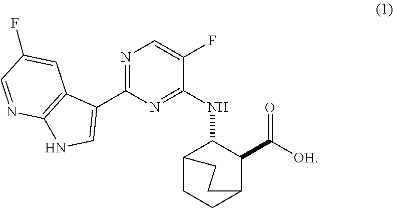 [SMILES: O=C(O)[C@H]1C2CCC(CC2)[C@@H]1Nc1nc(-c2c[nH]c3ncc(F)cc23)ncc1F]

For instance, the present invention provides a method of treating or reducing the severity of influenza virus infection comprising administering to a patient infected with influenza from about 200 mg to about 800 mg twice per day Compound (1) or a pharmaceutically acceptable salt thereof, wherein Compound (1) has the structure:

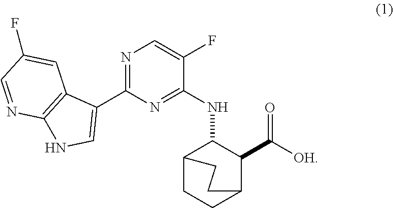 [SMILES: O=C(O)[C@H]1C2CCC(CC2)[C@@H]1Nc1nc(-c2c[nH]c3ncc(F)cc23)ncc1F]

In some embodiments, the patient is administered a crystalline form of the HCl salt of Compound (1).

In some embodiments, the patient is administered from about 250 mg to about 750 mg of Compound (1). For example, the patient is administered about 600 mg of Compound (1), or a pharmaceutically acceptable salt thereof, twice per day.

In some embodiments, Compound (1) or a pharmaceutically acceptable salt thereof is administered to the patient every day for 3 to 10 days.

In some embodiments, the influenza virus is influenza A virus.

Some embodiments further comprise administering an additional therapeutic agent (e.g., oseltamivir or a pharmaceutically acceptable salt thereof). Some embodiments further comprise administering from about 50 mg to about 100 mg of oseltamivir at least once per day. For example, some embodiments comprise administering about 75 mg of oseltamivir at least once per day.

Some embodiments further comprise co-administering about 75 mg of oseltamivir, or a pharmaceutically acceptable salt thereof, twice per day with said Compound (1) or pharmaceutically acceptable salt thereof.

The invention also relates to a method of treating or reducing the severity of influenza virus infection comprising administering to a patient a pharmaceutical combination comprising about 200 mg to about 800 mg Compound (1) or a pharmaceutically acceptable salt thereof, and about 50 mg to about 100 mg of oseltamivir or a pharmaceutically acceptable salt thereof.

For instance, the present invention also provides a method of treating or reducing the severity of influenza virus infection comprising administering to a patient infected with influenza a pharmaceutical combination comprising from about 200 mg to about 800 mg of Compound (1) or a pharmaceutically acceptable salt thereof, and from about 50 mg to about 100 mg of oseltamivir or a pharmaceutically acceptable salt thereof at least once per day, wherein Compound (1) has the structure:

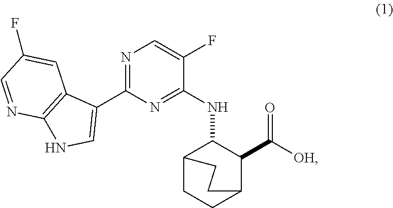 [SMILES: O=C(O)[C@H]1C2CCC(CC2)[C@@H]1Nc1nc(-c2c[nH]c3ncc(F)cc23)ncc1F]

and
wherein said administration is first effected within 48 to 96 hours of onset of at least one influenza symptom in said patient.

In some embodiments, administration is first effected within about 60 to about 96 hours of said onset of influenza symptom in said patient. For example, administration is first effected within about 72 to about 96 hours of said onset of influenza symptom in said patient. In another example, administration is first effected within about 72 hours of said onset of influenza symptoms in said patient.

In some embodiments, the influenza symptom includes at least one symptom selected from nasal congestion, sore throat, cough, aches, fatigue, headaches, and chills/sweats.

In some embodiments, the combination comprises from about 300 mg to about 600 mg of Compound (1) or a pharmaceutically acceptable salt thereof. For example, the combination comprises about 600 mg of Compound (1) or a pharmaceutically acceptable salt thereof.

In some embodiments, the combination comprises about 75 mg of oseltamivir or a pharmaceutically acceptable salt thereof.

In some embodiments, the combination comprises about 600 mg of Compound (1) or a pharmaceutically acceptable salt thereof and about 75 mg of oseltamivir or a pharmaceutically acceptable salt thereof.

In some embodiments, the combination comprises about 600 mg of Compound (1) or a pharmaceutically acceptable salt thereof and about 75 mg of oseltamivir or a pharmaceutically acceptable salt thereof and the combination is administered twice per day.

In some embodiments, the combination comprises about 600 mg of Compound (1) or a pharmaceutically acceptable salt thereof and about 75 mg of oseltamivir or a pharmaceutically acceptable salt thereof, the combination is administered twice per day, and said administration is first effected within about 72 hours to about 96 hours of onset of symptoms of said influenza.

In some embodiments, the combination comprises a crystalline form of the HCl salt of Compound (1).

In some embodiments, the oseltamivir or a pharmaceutically acceptable salt thereof is oseltamivir phosphate.

In some embodiments, the combination is administered to the patient every day for 3 to 10 days.

In some embodiments, the influenza virus is influenza A virus.

In some embodiments, administration is first effected after the patient's oxygen saturation level has fallen below 94%, as measured by pulse oximetry or after the patient has been administered supplemental oxygen.

The present invention also provides a kit for treating or reducing the severity of influenza virus infection comprising Compound (1) or a pharmaceutically acceptable salt thereof or a pharmaceutical composition comprising compound (1) or a pharmaceutically acceptable salt thereof and at least one leaflet comprising prescribing information, wherein said prescribing information comprises a method as described herein.

In some embodiments, the kit comprises a crystalline form of the HCl salt of Compound (1).

The invention also relates to a pharmaceutical combination comprising about 200 mg to about 800 mg Compound (1) or a pharmaceutically acceptable salt thereof, and about 50 mg to about 100 mg of oseltamivir or a pharmaceutically acceptable salt thereof, wherein oseltamivir has the structure:

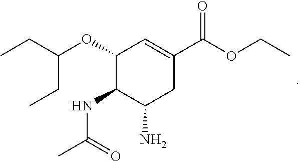 [SMILES: CCOC(=O)C1=C[C@@H](OC(CC)CC)[C@H](NC(C)=O)[C@@H](N)C1]

### BRIEF DESCRIPTION OF THE FIGURES

FIG. 1 describes the key design elements of a randomized, double-blind, placebo-controlled, parallel-group, multicenter clinical study.

FIG. 2 is a flow-chart detailing the participation by the subjects, including a breakdown of randomization, treatment, completion, discontinuation, and screening failures.

FIG. 3 is a graphical representation of the estimated LS Means and 95% CIs for viral load over time.

FIG. 4 is a Kaplan-Meier plot of time to resolution of influenza symptoms by treatment group.

FIG. 5 is a graph of the estimated survival curves of the time to resolution of the 7 primary influenza symptoms, using an accelerated failure time model, based on the mean baseline influenza symptom score and weighted average for stratum.

FIG. 6 is a bar graph providing percentages of subjects with viral load (qRT-PCR) categorized as negative (target not detected), positive (target detected), and ≥limit of quantification for each visit and treatment group.

FIG. 7 is a line graph providing the estimated survival curves for time to negativity of qRT-PCR based on the mean baseline viral load and weighted average for stratum.

FIG. 8 is a bar graph providing percentages of subjects with viral load (viral culture) categorized as negative and positive for each visit and treatment group.

FIG. 9 is a Kaplan-Meier plot of time to resolution of fever by treatment group.

### DETAILED DESCRIPTION OF THE INVENTION

The present invention provides mono and co-therapies that are useful for treating (e.g., reducing the symptoms) and/or preventing influenza virus infection in a patient.

I. DEFINITIONS AND COMMONLY USED ABBREVIATIONS

Commonly Used Abbreviations
AE adverse event
AUC area under the curve
bid twice daily
CI confidence interval
FAS Full Analysis Set
LS least square
qRT-PCR quantitative reverse transcriptase polymerase chain reaction
SAE serious adverse event
TEAE treatment-emergent adverse event
OST oseltamivir
Cmpd (1) Compound (1)
LoD Limit of Detection
LoQ Limit of Quantification

As used herein, “oseltamivir”, abbreviated as “OST”, refers to an acetamido cyclohexene compound having the structure

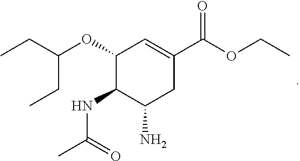 [SMILES: CCOC(=O)C1=C[C@@H](OC(CC)CC)[C@H](NC(C)=O)[C@@H](N)C1]

Oseltamivir is a neuraminidase inhibitor that is sold (in phosphate salt form) under the trade name Tamiflu®.

As used herein, an “excipient” is an inactive ingredient in a pharmaceutical composition. Examples of excipients include fillers or diluents, wetting agents (e.g., surfactants), binders, glidants, lubricants, disintegrants, or the like.

As used herein, a “disintegrant agent” is an excipient that hydrates a pharmaceutical composition and aids in tablet dispersion. Examples of disintegrant agents include sodium croscarmellose, polyplasdone (i.e., cross-linked polyvinylpyrollidone), sodium starch glycolate, or any combination thereof.

As used herein, a “diluent” or “filler” is an excipient that adds bulkiness to a pharmaceutical composition. Examples of fillers include lactose, sorbitol, celluloses, calcium phosphates, starches, sugars (e.g., mannitol, sucrose, or the like) or any combination thereof.

As used herein, a “wetting agent” or a “surfactant” is an excipient that imparts pharmaceutical compositions with enhanced solubility and/or wetability. Examples of wetting agents include sodium lauryl sulfate (SLS), sodium stearyl fumarate (SSF), polyoxyethylene 20 sorbitan mono-oleate (e.g., Tween®, or any combination thereof.

As used herein, a “binder” is an excipient that imparts a pharmaceutical composition with enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein, a “glidant” is an excipient that imparts a pharmaceutical compositions with enhanced flow properties. Examples of glidants include colloidal silica and/or talc.

As used herein, a “colorant” is an excipient that imparts a pharmaceutical composition with a desired color. Examples of colorants include commercially available pigments such as FD&C Blue # 1 Aluminum Lake, FD&C Blue #2, other FD&C Blue colors, titanium dioxide, iron oxide, and/or combinations thereof. Other colorants include commercially available pigments such as FD&C Green #3.

As used herein, a “lubricant” is an excipient that is added to pharmaceutical compositions that are pressed into tablets. The lubricant aids in compaction of granules into tablets and ejection of a tablet of a pharmaceutical composition from a die press. Examples of lubricants include magnesium stearate, stearic acid (stearin), hydrogenated oil, sodium stearyl fumarate, or any combination thereof.

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds.

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as analytical tools or probes in biological assays. Such compounds, especially deuterium (D) analogs, can also be therapeutically useful.

The compounds described herein are defined herein by their chemical structures and/or chemical names. Where a compound is referred to by both a chemical structure and a chemical name, and the chemical structure and chemical name conflict, the chemical structure is determinative of the compound's identity.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds in accordance with the present invention are provided in the form of a single enantiomer at least 95%, at least 97% and at least 99% free of the corresponding enantiomer.

In a further embodiment, the compounds in accordance with the present invention are in the form of the (+) enantiomer at least 95% free of the corresponding (−) enantiomer.

In a further embodiment, the compounds in accordance with the present invention are in the form of the (+) enantiomer at least 97% free of the corresponding (−) enantiomer.

In a further embodiment, the compounds in accordance with the present invention are in the form of the (+) enantiomer at least 99% free of the corresponding (−) enantiomer.

In a further embodiment, the compounds in accordance with the present invention are in the form of the (−) enantiomer at least 95% free of the corresponding (+) enantiomer.

In a further embodiment, the compounds in accordance with the present invention are in the form of the (−) enantiomer at least 97% free of the corresponding (+) enantiomer.

In a further embodiment the compounds in accordance with the present invention are in the form of the (−) enantiomer at least 99% free of the corresponding (+) enantiomer.

II. METHODS OF TREATMENT

One aspect of the present invention provides a method of treating or reducing the severity of influenza virus infection comprising administering to a patient infected with influenza from about 200 mg to about 800 mg at least once per day Compound (1) or a pharmaceutically acceptable salt thereof, wherein Compound (1) has the structure:

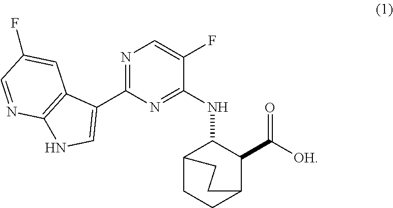 [SMILES: O=C(O)[C@H]1C2CCC(CC2)[C@@H]1Nc1nc(-c2c[nH]c3ncc(F)cc23)ncc1F]

In one embodiment of this aspect, the patient is administered from about 250 mg to about 750 mg Compound (1), or a pharmaceutically acceptable salt thereof.

In a further embodiment, the patient is administered from about 300 mg to about 600 mg Compound (1), or a pharmaceutically acceptable salt thereof.

In one embodiment, the patient is administered, twice per day for at least 3 or 5 days, about 600 mg of Compound (1), or a pharmaceutically acceptable salt thereof.

In another embodiment, the patient is administered a crystalline form of the HCl salt of Compound (1).

In one embodiment, Compound (1) or a pharmaceutically acceptable salt thereof is administered to the patient twice per day.

In another embodiment, Compound (1) or a pharmaceutically acceptable salt thereof is administered to the patient every day for 3 to 10 days.

In one embodiment, the influenza virus is influenza A virus.

In one embodiment, the method further comprises administering an additional therapeutic agent.

In a further embodiment, the additional therapeutic agent is a neuramidase inhibitor (e.g., oseltamivir or a pharmaceutically acceptable salt thereof).

In still a further embodiment, 50 mg to 100 mg of oseltamivir (e.g., Tamiflu®) is also administered at least once per day.

In one aspect, the invention includes a pharmaceutical combination comprising about 200 mg to about 800 mg Compound (1) or a pharmaceutically acceptable salt thereof, and about 50 mg to about 100 mg of oseltamivir or a pharmaceutically acceptable salt thereof, wherein oseltamivir has the structure:

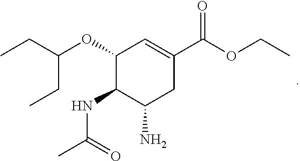 [SMILES: CCOC(=O)C1=C[C@@H](OC(CC)CC)[C@H](NC(C)=O)[C@@H](N)C1]

In one embodiment of this aspect, wherein the combination comprises about 250 mg to about 750 mg Compound (1) or a pharmaceutically acceptable salt thereof.

In a further embodiment, the combination comprises about 300 mg to about 600 mg Compound (1) or a pharmaceutically acceptable salt thereof.

In another embodiment, the Compound (1) or a pharmaceutically acceptable salt thereof is a crystalline form of the HCl salt of Compound (1).

In a further embodiment, the oseltamivir or a pharmaceutically acceptable salt thereof is oseltamivir phosphate.

In one embodiment, the combination comprises at least one tablet.

In another embodiment, the combination comprises two tablets, each contained in a single dosage package.

In a further embodiment, the single dosage package comprises a blister pack.

In one aspect, the invention includes a method of treating or reducing the severity of influenza virus infection comprising administering to a patient a combination described herein.

In one embodiment of this aspect, the combination is administered to the patient twice per day.

In one embodiment, the combination is administered to the patient every day for 3 to 10 days.

In another embodiment, the influenza virus is influenza A virus.

Crystalline Solid Forms of Compound (1)

Compound (1) represented by the following structural formula:

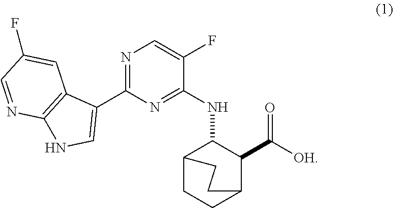 [SMILES: O=C(O)[C@H]1C2CCC(CC2)[C@@H]1Nc1nc(-c2c[nH]c3ncc(F)cc23)ncc1F]

Pharmaceutically acceptable salts of Compound (1) suitable for the present invention are described in WO 2010/148197 and WO 2015/073476.

Compound (1) can exist in or form different polymorphic forms. Polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds. Generally, different polymorphs can be characterized by analytical methods such as X-ray powder diffraction (XRPD) pattern, thermogravimetric analysis (TGA), and differential scanning calorimetry (DSC), or by its melting point, or other techniques known in the art. As used herein, the term “polymorphic form” includes solvates and neat polymorphic form that does not have any solvates.

As used herein, “Compound (1)” refers, generally, to the free base of Compound (1) and any hydrates thereof, including any polymorphic forms thereof “HCl salt of Compound (1)” means a HCl salt of the free base compound, and “tosylate salt of Compound (1)” means a tosylate salt of the free base compound. It is noted that Compound (1) and salts of Compound (1) can be solvated or non-solvated unless specified otherwise. Also, it is noted Compound (1) and salts of Compound (1) can be crystalline or amorphous unless specified otherwise.

In one embodiment, the present invention is directed to a crystalline form of an HCl salt of Compound (1), e.g., polymorphic Form A of HCl salt of Compound (1)•½ H₂O. This form is a polymorphic form of HCl salt of Compound (1) that includes water as a solvate in a half equivalent per Compound (1). In one specific embodiment, Form A of HCl salt of Compound (1)•½ H₂O is characterized by one or more peaks corresponding to 2-theta values measured in degrees of 10.5, 5.2, 7.4, and 18.9 (±0.2 degrees) in an X-ray powder diffraction pattern. In another specific embodiment, Form A of HCl salt of Compound (1)•½ H₂O is further characterized by one or more peaks corresponding to 2-theta values measured in degrees of 25.2±0.2, 16.5±0.2, 18.1±0.2, and 23.0±0.2 in an X-ray powder diffraction pattern. The XRPD patterns mentioned herein are obtained at room temperature using Cu K alpha radiation. In yet another specific embodiment, the polymorphic Form A of HCl salt of Compound (1)•½ H₂O is characterized as having one or more characteristic peaks at 29.2, 107.0, 114.0, and 150.7 (±0.3 ppm) in a C¹³ SSNMR spectrum. In yet another specific embodiment, the polymorphic Form A of HCl salt of Compound (1)•½ H₂O is further characterized as having one or more characteristic peaks at 22.1, 24.6, 47.7, and 54.8 (±0.3 ppm) in a C¹³ SSNMR spectrum.

In one embodiment, the present invention is directed to polymorphic Form F of HCl salt of Compound (1)•3H₂O. This form is a polymorphic form of HCl salt of Compound (1) that includes water as a solvate in three equivalents per Compound (1). In one specific embodiment, Form F of HCl salt of Compound (1)•3H₂O is characterized by one or more peaks corresponding to 2-theta values measured in degrees of 7.1, 11.9, 19.2, and 12.4 (±0.2) in an X-ray powder diffraction pattern. In another specific embodiment, Form F of HCl salt of Compound (1)•3H₂O is further characterized by one or more peaks corresponding to 2-theta values measured in degrees of 16.4, 21.8, and 23.9 (±0.2) in an X-ray powder diffraction pattern. These XRPD patterns are obtained at room temperature using Cu K alpha radiation. In yet another specific embodiment, the polymorphic Form F of HCl salt of Compound (1)•3H₂O is characterized by peaks at 20.7, 27.4, 104.8, 142.5, 178.6 (±0.3 ppm) in a C¹³ SSNMR spectrum. In yet another specific embodiment, the polymorphic Form F of HCl salt of Compound (1)•3H₂O is further characterized by one or more peaks corresponding to 154.3, 20.3, 132.3, and 21.1 (±0.3 ppm) in a C¹³ SSNMR spectrum.

In one embodiment, the present invention is directed to polymorphic Form D of HCl salt of Compound (1). This form is a non-solvated form of HCl salt of Compound (1). In one specific embodiment, Form D of HCl salt of Compound (1) is characterized by one or more peaks corresponding to 2-theta values measured in degrees of 5.8, 17.1, and 19.5 (±0.2) in an X-ray powder diffraction pattern. In another specific embodiment, Form D of HCl salt of Compound (1) is characterized by one or more peaks corresponding to 2-theta values measured in degrees of 5.3, 10.5, and 15.9 (±0.2) in an X-ray powder diffraction pattern. These XRPD patterns are obtained at room temperature using Cu K alpha radiation. In yet another specific embodiment, Form D of HCl salt of Compound (1) is characterized as having peaks at 29.4, 53.4, 113.3, 135.4, 177.8 (±0.3 ppm) in a C¹³ SSNMR spectrum. In yet another specific embodiment, Form D of HCl salt of Compound (1) is further characterized by one or more peaks corresponding to 22.9, 23.9, 26.0, and 31.6 (±0.3 ppm) in a C¹³ SSNMR spectrum.

In one embodiment, the present invention is directed to polymorphic Form A of Compound (1). This form is a non-solvated, free base form of Compound (1). In one specific embodiment, Form A of Compound (1) is characterized by one or more peaks corresponding to 2-theta values measured in degrees of 15.5, 18.9, and 22.0 (±0.2) in an X-ray powder diffraction pattern. In another specific embodiment, Form A of Compound (1) is further characterized by one or more peaks corresponding to 2-theta values measured in degrees of 11.8, 16.9, 25.5, and 9.1 (±0.2) in an X-ray powder diffraction pattern. These XRPD patterns are obtained at room temperature using Cu K alpha radiation. In yet another specific embodiment, Form A of Compound (1) is characterized as having peaks at 21.0, 28.5, 50.4, 120.8, 138.5, and 176.2 (±0.3 ppm) in a C¹³ SSNMR spectrum. In yet another specific embodiment, Form A of Compound (1) is characterized as having peaks at 30.1, 25.9, 22.8, and 25.0 (±0.3 ppm) in a C¹³ SSNMR spectrum.

In one embodiment, the present invention is directed to polymorphic Form A of tosylate salt of Compound (1). This form is a non-solvated form of tosylate salt of Compound (1). In one specific embodiment, Form A of tosylate salt of Compound (1) is characterized by one or more peaks corresponding to 2-theta values measured in degrees of 7.2, 9.3, 13.7, 14.3, 14.7, 16.9, 18.7, 26.3, and 26.9 (±0.2) in an X-ray powder diffraction pattern. In another specific embodiment, Form A of tosylate salt of Compound (1) is further characterized by one or more peaks corresponding to 2-theta values measured in degrees of 6.0, 28.0, and 27.5 (±0.2) in an X-ray powder diffraction pattern. The XRPD patterns are obtained at room temperature using Cu K alpha radiation.

In another embodiment, the present invention is directed to methods of preparing Form A of HCl salt of Compound (1)•½ H₂O, Form F of HCl salt of Compound (1)•3H₂O, Form D of HCl salt of Compound (1), Form A of Compound (1), and Form A of tosylate salt of Compound (1).

Form A of HCl salt of Compound (1)•½ H₂O can be prepared by employing mixing (e.g., stirring) hydrogen chloride (HC1) with Compound (1). Compound (1) can be solvated, non-solvated, amorphous, or crystalline. A solution, slurry, or suspension of Compound (1) can be mixed with HCl in a solvent system that includes water and one or more organic solvents, wherein the solvent system has a water activity of equal to, or greater than, 0.05 and equal to, or less than, 0.85, i.e., 0.05-0.85. The term “water activity” or “a_{w}”, as used herein, means a measure of the energy status of water in a solvent system. It is defined as the vapor pressure of a liquid divided by that of pure water at the same temperature. Specifically, it is defined as

$${a}_{w} = \frac{p}{{p}_{o}} ,$$

where p is the vapor pressure of water in the substance, and pₒ is the vapor pressure of pure water at the same temperature, or as a_{w}=l_{w}×x_{w}, where l_{w} is the activity coefficient of water and xₒ is the mole fraction of water in the aqueous fraction. For example, pure water has a water activity value of 1.0. Water activity values can typically be obtained by either a capacitance hygrometer or a dew point hygrometer. Various types of water activity measuring instruments are also commercially available. Alternatively, water activity values of mixtures of two or more solvents can be calculated based on the amounts of the solvents and the known water activity values of the solvents.

An example of crystalline Compound (1) includes Form A of Compound (1). Examples of solvates of Compound (1) include solvates of 2-MeTHF, N,N-dimentylacetamide, N,N-dimethylformamide, methanol, xylene, acetone, 2-butanol, methyl acetate, 1-pentanol, 2-propanol, tetrahydrofuran, methyl tetrahydrofuran, dimethylacetamide N,N-dimethylformamide 1,4-dioxane, 1-pentanol, 2-methy-1-propanol, methylethyl ketone, 3-methyl-1-butanol, heptane, ethyl formate, 1-butanol, acetic acid, and ethylene glycol. In a specific embodiment, solvates of 2-MeTHF (e.g., Compound (1). 1(2-MeTHF)) are employed.

The solvent systems suitable for the preparation of Form A of HCl salt of Compound (1)•½ H₂O can be comprised of a large variety of combinations of water and organic solvents where the water activity of the solvent systems is equal to, or greater than, 0.05 and equal to, or less than, 0.85 (0.05-0.85). In a specific embodiment, the value of the water activity is 0.4-0.6. Suitable organic solvents include Class II or Class III organic solvents listed in the International Conference on Harmonization Guidelines. Specific examples of suitable Class II organic solvents include chlorobenzene, cyclohexane, 1,2-dichloroethene, dichloromethane, 1,2-dimethoxyethane, N,N-dimentylacetamide, N,N-dimethylformamide, 1,4-dioxane, 2-ethoxyethanol, formamide, hexane, 2-methoxyethanol, methylbutyl ketone, methylcyclohexane, N-methylpyrrolidone, nitromethane, pyridine, sulfolane, tetrahydrofuran (THF), tetralin, tolune, 1,1,2-trichloroethene and xylene. Specific examples of suitable Class III organic solvents include: acetic acid, acetone, anisole, 1-butanol, 2-butanol, butyl acetate, tert-butylmethyl ether, cumene, heptane, isobutyl acetate, isopropyl acetate, methyl acetate, 3-methyl-1-butanol, methylethyl ketone, methylisobutyl ketone, 2-methyl-1-propanol, ethyl acetate, ethyl ether, ethyl formate, pentane, 1-pentanol, 1-propanol, 2-propanol and propyl acetate. In one specific embodiment, the organic solvents of the solvent system are selected from the group consisting of chlorobenzene, cyclohexane, 1,2-dichloroethane, dichloromethane, 1,2-dimethoxyethane, hexane, 2-methoxyethanol, methylbutyl ketone, methylcyclohexane, nitromethane, tetralin, xylene, toluene, 1,1,2-trichloroethane, acetone, anisole, 1-butanol, 2-butanol, butyl acetate, tert-butylmethyl ether, cumene, ethanol, ethyl acetate, ethyl ether, ethyl formate, heptane, isobutyl acetate, isopropyl acetate, methyl acetate, 3-methyl-1-butanol, methylethyl ketone, 2-methy-1-propanol, pentane, 1-propanol, 1-pentanol, 2-propanol, propyl acetate, tetrahydrofuran, and methyl tetrahydrofuran. In another specific embodiment, the organic solvents of the solvent system are selected from the group consisting of 2-ethoxyethanol, ethyleneglycol, methanol, 2-methoxyethanol, 1-butanol, 2-butanol, 3-methyl-1-butanol, 2-methyl-1-propanol, ethanol, 1-pentanol, 1-propanol, 2-propanol, methylbutyl ketone, acetone, methylethyl ketone, methylisobutyl ketone, butyl acetate, isobutyl acetate, isopropyl acetate, methyl acetate, ethyl acetate, propyl acetate, pyridine, toluene, and xylene. In yet another embodiment, the organic solvents are selected from the group consisting of acetone, n-propanol, isopropanol, iso-butylacetate, and acetic acid. In yet another embodiment, the organic solvents are selected from the group consisting of acetone and isopropanol. In yet another specific embodiment, the solvent system includes water an acetone. In yet another specific embodiment, the solvent system includes water an isopropanol.

The preparation of Form A of HCl salt of Compound (1)•½ H₂O can be performed at any suitable temperature. Typically, it is performed at a temperature of 5° C.-75° C. In a specific embodiment, it is performed at a temperature of 15° C.-75° C. In another specific embodiment, it is performed at a temperature of 15° C.-60° C. In yet another specific embodiment, it is performed at a temperature of 15° C.-35° C. In yet another specific embodiment, the preparation is performed at 5° C.-75° C. in a solvent system having a water activity value of 0.4-0.6. In yet another specific embodiment, the preparation is performed at a temperature of 15° C.-75° C. in a solvent system having a water activity value of 0.4-0.6. In yet another specific embodiment, the preparation is performed at a temperature of 15° C.-60° C. in a solvent system having a water activity value of 0.4-0.6. In yet another specific embodiment, the preparation is performed at 15° C.-35° C. in a solvent system having a water activity value of 0.4-0.6.

The hydrogen chloride (HCl) can be introduced as a solution or gas. One example, a suitable hydrogen chloride source is an aqueous solution of hydrogen chloride comprising 30-40 wt % (e.g., 34 wt %-38 wt %) of HCl by weight of the aqueous solution.

Form F of HCl salt of Compound (1)•3H₂O can be prepared by mixing HCl and Compound (1) in a solvent system that includes water or that includes water and one or more organic solvents, wherein the solvent system has a water activity of equal to, or greater than, 0.9 (≥0.9). The mixture can be a solution, slurry, or suspension. Compound (1) can be solvated, non-solvated, amorphous, or crystalline. Alternatively, it can be prepared by stirring Form A of HCl salt of Compound (1)•½ H₂O in a solvent system that includes water or that includes water and one or more organic solvents, wherein the solvent system has a water activity of equal to, or greater than, 0.9. Typically, pure water has a water activity value of 1.0. Accordingly, a solvent system having a water activity of 0.9-1.0 can be suitable for the preparation of Form F of HCl salt of Compound (1)•3H₂O. In a specific embodiment, the mixing or stirring is performed at an ambient temperature (18° C.-25° C.). In another specific embodiment, the mixing or stirring is performed at a temperature of 15° C.-30° C. In another specific embodiment, the mixing or stirring is performed at a temperature of 20° C.-28° C. (e.g., 25° C.). Suitable organic solvents, including specific examples, for the formation of Form F of HCl salt of Compound (1)•3H₂O are as described above for Form A of HCl salt of Compound (1)•½ H₂O. In yet another specific embodiment, the solvent system includes water an acetone. In yet another specific embodiment, the solvent system includes water an isopropanol.

Form D of HCl salt of Compound (1) can be prepared by dehydrating Form A of HCl salt of Compound (1)•½ H₂O. The dehydration can be done by any suitable means, such as heating or dry nitrogen purge, or both.

Form A of Compound (1) can be prepared by (a) stirring a mixture of amorphous Compound (1) or a solvate of Compound (1) (such as a 2-MeTHF solvate of Compound (1)) in a solvent system that includes water and ethanol. The mixture can be a solution or slurry. In a specific embodiment, the stirring step is performed at a temperature in a range of 18° C. to 90° C. In another specific embodiment, the stirring step (a) is performed at a refluxing temperature of the solvent system. In another specific embodiment, the solvent system includes 5 wt % to 15 wt % of water by weight of the solvent system. Examples of solvates of Compound (1) are as described above. In a specific embodiment, solvates of 2-MeTHF (e.g., Compound (1)•1(2-MeTHF)) are employed.

In another embodiment, the methods of preparing Form A of Compound (1) further comprises: (b) stirring amorphous form of Compound (1) in nitromethane to form crystalline seed of Form A of Compound (1); and (c) adding the crystalline seed of Form A of Compound (1) to the resulting mixture of the mixing step (a). In a specific embodiment, the methods further comprises: (b) stirring the amorphous form of Compound (1) in nitromethane to form crystalline seed of Form A of Compound (1); (c) cooling the resulting mixture of the mixing step (a) to a temperature in a range of 18° C. to 60° C. (e.g., 50° C.-55° C. or 55° C.); and (d) adding the crystalline seed of Form A of Compound (1) to the resulting mixture step (c). In another specific embodiment, the methods further comprise adding water, prior to the addition of crystalline seed of Form A of Compound (1), to the resulting mixture that has gone through the refluxing step in an amount to have the resulting solvent system include water by 15-25 wt % after the addition of water. In yet another specific embodiment, the methods further comprises adding water to the mixture that includes crystalline seed of Form A of Compound (1) in an amount to have the resulting solvent system include water by 35-45 wt % after the addition of water. In yet another specific embodiment, the methods further comprises cooling the mixture that includes crystalline seed of Form A of Compound (1), after the addition of water, to a temperature of 0° C.-10° C.

In one specific embodiment, the crystalline seed of Form A of Compound (1) can be prepared by 2-MeTHF solvate of Compound (1) in nitromethane. In one embodiment, the solvent system for the refluxing step includes 5-15 wt % (e.g., 8 wt %, 10 wt %, or 12 wt %) of water by weight of the solvent system.

Form A of tosylate salt of Compound (1) can be prepared by stirring a mixture of amorphous Compound (1) or a solvate of Compound (1) ((such as a 2-MeTHF solvate of Compound (1)), p-toluenesulfonic acid, and a solvent system that includes acetonitrile. In a specific embodiment, the mixing or stirring step is performed at an ambient temperature. In another specific embodiment, the mixing or stirring step is performed at a temperature of 15-30° C. In another specific embodiment, the mixing or stirring step is performed at a temperature of 20-30° C. (e.g., 25° C.). Suitable examples of solvates of Compound (1), including specific examples, are as described above for the preparation of Form A of Compound (1).

In yet another embodiment, the invention is directed to 2-MeTHF solvates of Compound (1). In one specific embodiment, the solvates include 0.5-1.5 equivalents of 2-MeTHF per Compound (1), such as 1 equivalent of 2-MeTHF per Compound (1). In one specific embodiment, the solvates include 1 equivalent of 2-MeTHF and characterized as having an XRPD pattern with characteristic peaks expressed in 2-theta±0.2 at the following positions at 8.4, 9.7, 16.7, 16.9, 17.4, 21.0, 22.3, and 25.7.

In yet another embodiment, the invention encompasses amorphous forms of Compound (1) and pharmaceutically acceptable salts thereof, such as amorphous HCl salt of Compound (1) and amorphous Compound (1). In yet another embodiment, the invention also encompasses Form B of Compound (1) hydrate. Form B of Compound (1) hydrate is isomorphic with Form A of Compound (1), showing the same XRPD peaks as those for Form A of Compound (1), but formed in the presence of water, for example, in a system having a water activity greater than 0.6, such as 0.6-1.0, at ambient temperature.

The present invention encompasses the polymorphic forms of Compound (1) described above in isolated, pure form, or in a mixture as a solid composition when admixed with other materials, for example the other forms (i.e., amorphous form, Form A of Compound (1), etc.) of Compound (I) or any other materials.

In one aspect, the present invention provides polymorphic forms, such as Form A of HCl salt of Compound (1)•½ H₂O, Form F of HCl salt of Compound (1)•3H₂O, Form D of HCl salt of Compound (1), Form A of Compound (1), Form B of Compound (1) hydrate, and Form A of tosylate salt of Compound (1), in isolated solid form. In yet another aspect, the present invention provides amorphous form of Compound (1) and pharmaceutically acceptable salts thereof, such as amorphous HCl salt of Compound (1) and amorphous Compound (1), in isolated solid form.

In a further aspect, the present invention provides polymorphic forms, such as Form A of HCl salt of Compound (1)•½ H₂O, Form F of HCl salt of Compound (1)•3H₂O, Form D of HCl salt of Compound (1), Form A of Compound (1), Form B of Compound (1) hydrate and Form A of tosylate salt of Compound (1), in pure form. The pure form means that the particular polymorphic form comprises over 95% (w/w), for example, over 98% (w/w), over 99% (w/w %), over 99.5% (w/w), or over 99.9% (w/w). In another further aspect, there is provided amorphous forms of Compound (1) or pharmaceutically acceptable salts thereof in pure form. The pure form means that the amorphous form is over 95% (w/w), for example, over 98% (w/w), over 99% (w/w %), over 99.5% (w/w), or over 99.9% (w/w).

More specifically, the present invention provides that each of the polymorphic forms in the form of a composition or a mixture of the polymorphic form with one or more other crystalline, solvate, amorphous, or other polymorphic forms or their combinations thereof. For example, in one embodiment, the composition comprises Form A of HCl salt of Compound (1)•½ H₂O along with one or more other polymorphic forms of Compound (1), such as amorphous form, solvates, Form D of HCl salt of Compound (1), Form F of HCl salt of Compound (1)•3H₂O, Form A of Compound (1), and/or other forms or any combination thereof. Similarly, in another embodiment, the composition comprises Form F of HCl salt of Compound (1)•3H₂O along with one or more other polymorphic forms of Compound (1), such as amorphous form, solvates, Form A of HCl salt of Compound (1)•½ H₂O, Form D of HCl salt of Compound (1), Form A of Compound (1), and/or other forms or their combinations thereof. Similarly, in another embodiment, the composition comprises Form D of HCl salt of Compound (1) along with one or more other polymorphic forms of Compound (1), such as amorphous form, solvates, Form A of HCl salt of Compound (1)•½ H₂O, Form F of HCl salt of Compound (1)•3H₂O, Form A of Compound (1), and/or other forms or their combinations thereof. In yet another embodiment, the composition comprises Form A of Compound (1) along with one or more other polymorphic forms of Compound (1), such as amorphous form, hydrates, solvates, and/or other forms or their combinations thereof. In yet another embodiment, the composition comprises Form A of tosylate salt of Compound (1) along with one or more other polymorphic forms of Compound (1), such as amorphous form, hydrates, solvates, and/or other forms or their combinations thereof. More specifically, the composition may comprise from trace amounts up to 100% of the specific polymorphic form or any amount, for example, in a range of 0.1%-0.5%, 0.1%-1%, 0.1%-2%, 0.1%-5%, 0.1%-10%, 0.1%-20%, 0.1%-30%, 0.1%-40%, or 0.1%-50% by weight based on the total amount of Compound (1) in the composition. Alternatively, the composition may comprise at least 50%, 60%, 70%, 80%, 90%, 95%, 97%, 98%, 99%, 99.5% or 99.9% by weight of specific polymorphic form based on the total amount of Compound (1) in the composition.

Pharmaceutical Compositions

Fillers (or diluents) typically include microcrystalline celluloses (e.g., Avicel® PH 101), lactoses, sorbitols, celluloses, calcium phosphates, starches, sugars (e.g., mannitol, sucrose, or the like), or any combination thereof. Specific examples of the fillers include microcrystalline celluloses and lactoses. Specific examples of microcrystalline celluloses include commercially available Avicel® series, such as microcrystalline celluloses having a particle size of 200 mesh over 70% and a particle size of 65 mesh less than 10% (e.g., Avicel® PH 101). Other specific examples of microcrystalline celluloses are silicified microcrystalline celluloses, such as commercially available Prosolv® series (e.g., Prosolv® SMCC 50). A specific example of lactose suitable for the invention includes lactose monohydrate. Typical amounts of the fillers relative to the total weight of the pharmaceutical composition may be 5 wt % to 95 wt %, 20 wt % to 80 wt %, or 25 wt % to 50 wt %.

In one embodiment, the pharmaceutical compositions of the invention further comprise 1 wt % to 10 wt % of a disintegrant agent by the weight of the pharmaceutical composition. In one specific embodiment, 3 wt % to 7 wt % of a disintegrant agent by the weight of the pharmaceutical composition is employed.

Disintegrants typically enhance the dispersal of pharmaceutical compositions. Examples of disintegrants include croscarmelloses (e.g., croscarmellose sodium), crospovidones, starch (e.g., corn starch, potato starch), metal starch glycolates (e.g., sodium starch glycolate), and any combination thereof. Specific examples of disintegrants include croscarmellose sodium (e.g., Ac-Di-Sol®) and sodium starch glycolate. Typical amounts of the disintegrants relative to the total weight of the pharmaceutical composition may be 1 wt % to 10 wt %, 3 wt % to 7 wt %, or 1 wt % to 5 wt % of the pharmaceutical compositions.

In another embodiment, the pharmaceutical compositions of the invention further comprise 0.1 wt % to 7wt %, preferably 0.2wt % to 5 wt % of a binder by the weight of the pharmaceutical composition. In one specific embodiment, 0.5 wt % to 2 wt % of a binder by the weight of the pharmaceutical composition is employed.

Binders typically include agents used while making granules of the active ingredient by mixing it with diluent fillers. Exemplary binders include polyvinyl pyrrolidones, starch (e.g., pregelatinized starch), sugar, microcrystalline celluloses, modified celluloses (e.g., hydroxy propyl methyl celluloses (HPMC), hydroxy propyl celluloses (HPC), hydroxy ethyl celluloses (HEC), and any combination thereof. Specific examples of the binders include polyvinyl pyrrolidones (PVP). An example of HPC includes a low viscosity polymer, HPC-SL. PVP is commonly characterized by the so-called “K-value”, which is a useful measure of the polymeric composition's viscosity. PVP can be commercially purchased (e.g., Tokyo Chemical Industry Co., Ltd.) under the trade name of Povidone® K12, Povidone® K17, Povidone® K25, Povidone® K30, Povidone® K60, and Povidone® K90. Specific examples of PVP include soluble spray dried PVP. A more specific example includes PVP having an average molecular weight of 3,000 to 4,000, such as Povidone® K12 having an average molecular weight of 4,000. PVP can be used in either wet or dry state. Typical amounts of the binders relative to the total weight of the pharmaceutical composition may be 0.1 wt % to 5 wt %, or 0.5 wt % to 2 wt %.

In yet another embodiment, the pharmaceutical compositions of the invention further comprise 0.5 wt % to 5 wt % of a lubricant by the weight of the pharmaceutical composition. In one specific embodiment, 0.5 wt % to 3 wt % or 1 wt % to 3 wt % of a lubricant by the weight of the pharmaceutical composition is employed.

Lubricants typically improve the compression and ejection of pharmaceutical compositions from, e.g., a die press. Exemplary lubricants include magnesium stearate, stearic acid (stearin), hydrogenated oil, sodium stearyl fumarate, and any combinations thereof. A specific example of the lubricants includes sodium stearyl fumarate. Another specific example of the lubricants includes magnesium stearate. Typical amounts of the lubricants relative to the total weight of the pharmaceutical composition may be 0.1 wt % to 7wt %, 0.3 wt % to 5 wt %, 0.5 wt % to 3 wt %, or 1 wt % to 3 wt %.

In some embodiments, a wetting agent can be employed in the pharmaceutical compositions of the invention. Wetting agents typically include surfactants, such as non-ionic surfactants and anionic surfactants. Wetting agents suitable for the present invention generally enhance the solubility of pharmaceutical compositions. Exemplary surfactants include sodium lauryl sulfate (SLS), polyoxyethylene sorbitan fatty acids (e.g., Tween®), sorbitan fatty acid esters (e.g., Spans®), sodium dodecylbenzene sulfonate (SDBS), dioctyl sodium sulfosuccinate (Docusate), dioxycholic acid sodium salt (DOSS), Sorbitan Monostearate, Sorbitan Tristearate, Sodium N-lauroylsarcosine, Sodium Oleate, Sodium Myristate, Sodium Stearate, Sodium Palmitate, Gelucire 44/14, ethylenediamine tetraacetic acid (EDTA), Vitamin E d-alpha tocopheryl polyethylene glycol 1000 succinate (TPGS), Lecithin, MW 677-692, Glutanic acid monosodium monohydrate, Labrasol, PEG 8 caprylic/capric glycerides, Transcutol, diethylene glycol monoethyl ether, Solutol HS-15, polyethylene glycol/hydroxystearate, Taurocholic Acid, copolymers of polyoxypropylene and polyoxyethylene (e.g., poloxamers also known and commercially available under Pluronics®, such as, Pluronic® L61, Pluronic® F68, Pluronic® F108, and Pluronic® F127), saturated polyglycolized glycerides (Gelucirs®), and any combinations thereof. Specific examples include sodium lauryl sulfate, which is an anionic surfactant; and copolymers of polyoxypropylene and polyoxyethylene which are non-ionic surfactants. Specific examples of the copolymers of polyoxypropylene and polyoxyethylene include poloxamers, such as poloxamer with a polyoxypropylene molecular mass of 1,800 g/mol and a 80% polyoxyethylene content (e.g., poloxamer 188). Typical amounts of the wetting agents relative to the total weight of the pharmaceutical composition may be 0.25 wt % to 10 wt %, or 1 wt % to 5 wt %.

The wetting agents, binders, disintegrants, lubricants, and fillers suitable for the invention are compatible with the ingredients of the pharmaceutical compositions of the invention—for example, they do not substantially reduce the chemical stability.

In one specific embodiment, the pharmaceutical compositions of the invention comprise: a) 20 wt % to 80 wt % of a HCl salt of Compound (1)•xH₂O by the weight of the pharmaceutical composition; b) 1 wt % to 10 wt % of a disintegrant agent by the weight of the pharmaceutical composition; and c) 20 wt % to 80 wt % of a filler by the weight of the pharmaceutical composition. In another specific embodiment, the pharmaceutical compositions of the invention comprise: a) 20 wt % to 80 wt % of a HCl salt of Compound (1)•xH₂O by the weight of the pharmaceutical composition; b) 1 wt % to 10 wt % of a disintegrant agent by the weight of the pharmaceutical composition; c) 0.1 wt % to 7 wt %, 0.2 wt % to 5 wt % of a binder by the weight of the pharmaceutical composition; and d) 20 wt % to 80 wt % of a filler by the weight of the pharmaceutical composition. In yet another specific embodiment, the pharmaceutical compositions of the invention comprise: a) 20 wt % to 80 wt % of a HCl salt of Compound (1)•xH₂O by the weight of the pharmaceutical composition; b) 1 wt % to 10 wt % of a disintegrant agent by the weight of the pharmaceutical composition; c) 0.1 wt % to 7 wt %, 0.2 wt % to 5 wt % of a binder by the weight of the pharmaceutical composition; d) 20 wt % to 80 wt % of a filler by the weight of the pharmaceutical composition; and e) 0.5 wt % to 7 wt %, 0.6 wt % to 5 wt % of a lubricant by the weight of the composition. Examples, including specific examples, of the fillers, disintegrant agents, binders, and lubricants are as described above.

In yet another specific embodiment, the pharmaceutical compositions of the invention comprise: a) 35 wt % to 75 wt % of a HCl salt of Compound (1)•xH₂O by the weight of the pharmaceutical composition; b) 1 wt % to 7 wt % of a disintegrant agent by the weight of the pharmaceutical composition, wherein the disintegrant is selected from a croscarmellose, a crospovidone, a metal starch glycolate or a starch, or any combination thereof; c) 0.5 wt % to 2 wt % of a binder by the weight of the pharmaceutical composition, wherein the binder is selected from a polyvinyl pyrrolidone, a starch, a sugar, a microcrystalline cellulose, a hydroxy propyl methyl cellulose, a hydroxy propyl cellulose, or a hydroxy ethyl cellulose, or any combination thereof; d) 25 wt % to 50 wt % of a filler by the weight of the pharmaceutical composition; wherein the filler is selected from a microcrystalline cellulose, a lactose, a sorbitol, a cellulose, a calcium phosphate, a starch, or a sugar, or any combination thereof; and e) 0.5 wt % to 3 wt % of a lubricant by the weight of the composition, wherein the lubricant is selected from a metal stearate and/or a metal stearyl fumarate. Specific examples of the fillers, disintegrant agents, binders, and lubricants are as described above.

In yet another specific embodiment, the pharmaceutical compositions of the invention comprise: a) 35 wt % to 75 wt % of a HCl salt of Compound (1)•xH₂O by the weight of the pharmaceutical composition, wherein x is from 0 to 3 (e.g., 0.5); b) 3 wt % to 7 wt % of a croscarmellose by the weight of the pharmaceutical composition; c) 0.5 wt % to 2 wt % a polyvinyl pyrrolidone by the weight of the pharmaceutical composition; d) 25 wt % to 50 wt % of a filler by the weight of the pharmaceutical composition; wherein the filler includes a microcrystalline cellulose and a lactose; and e) 0.5 wt % to 3 wt % of a metal stearyl fumarate by the weight of the composition. Specific examples of the fillers, disintegrant agents, binders, and lubricants are as described above.

In yet another specific embodiment, the pharmaceutical compositions of the invention comprise: a) 35 wt % to 75 wt % of a HCl salt of Compound (1)•xH₂O by the weight of the pharmaceutical composition, wherein x is from 0 to 3 (e.g., 0.5); b) 3 wt % to 7 wt % of a croscarmellose by the weight of the pharmaceutical composition; c) 0.5 wt % to 2 wt % of a polyvinyl pyrrolidone by the weight of the pharmaceutical composition; d) 25 wt % to 50 wt % of a filler by the weight of the pharmaceutical composition; wherein the filler includes a microcrystalline cellulose and a lactose; and e) 0.5 wt % to 3 wt % of sodium stearyl fumarate by the weight of the composition. Specific examples of the fillers, disintegrant agents, binders, and lubricants are as described above.

In yet another specific embodiment, the pharmaceutical compositions of the invention comprise: a) 35 wt % to 65 wt % of a HCl salt of Compound (1)•xH₂O by the weight of the pharmaceutical composition, wherein x is from 0 to 3 (e.g., 0.5); b) 3 wt % to 7 wt % of croscarmellose sodium by the weight of the pharmaceutical composition; c) 0.5 wt % to 2 wt % of a polyvinyl pyrrolidone having an average molecular weight of 3,000 to 5,000 by the weight of the pharmaceutical composition; d) 30 wt % to 40 wt % of a microcrystalline cellulose by the weight of the pharmaceutical composition; e) 5 wt % to 10 wt % of lactose monohydrate by the weight of the pharmaceutical composition; and f) 1 wt % to 3 wt % of sodium stearyl fumarate by the weight of the composition.

In one further specific embodiment, the pharmaceutical compositions of the invention comprise: a) 20 wt % to 80 wt % of Form A of HCl salt of Compound (1)•½ H₂O by the weight of the pharmaceutical composition; b) 1 wt % to 10 wt % of a disintegrant agent by the weight of the pharmaceutical composition; and c) 20 wt % to 80 wt % of a filler by the weight of the pharmaceutical composition. In another further specific embodiment, the pharmaceutical compositions of the invention comprise: a) 20 wt % to 80 wt % of Form A of HCl salt of Compound (1)•½ H₂O by the weight of the pharmaceutical composition; b) 1 wt % to 10 wt % of a disintegrant agent by the weight of the pharmaceutical composition; c) 0.1 wt % to 5 wt % of a binder by the weight of the pharmaceutical composition; and d) 20 wt % to 80 wt % of a filler by the weight of the pharmaceutical composition. In yet another further specific embodiment, the pharmaceutical compositions of the invention comprise: a) 20 wt % to 80 wt % of Form A of HCl salt of Compound (1)•½ H₂O by the weight of the pharmaceutical composition; b) 1 wt % to 10 wt % of a disintegrant agent by the weight of the pharmaceutical composition; c) 0.1 wt % to 5 wt % of a binder by the weight of the pharmaceutical composition; d) 20 wt % to 80 wt % of a filler by the weight of the pharmaceutical composition; and e) 0.5 wt % to 5 wt % of a lubricant by the weight of the composition. Examples, including specific examples, of the fillers, disintegrant agents, binders, and lubricants are as described above.

In yet another further specific embodiment, the pharmaceutical compositions of the invention comprise: a) 35 wt % to 75 wt % of Form A of HCl salt of Compound (1)•½ H₂O by the weight of the pharmaceutical composition; b) 1 wt % to 7 wt % of a disintegrant agent by the weight of the pharmaceutical composition, wherein the disintegrant is selected from a croscarmellose, a crospovidone, a metal starch glycolate or a starch, or any combination thereof; c) 0.5 wt % to 2 wt % of a binder by the weight of the pharmaceutical composition, wherein the binder is selected from a polyvinyl pyrrolidone, a starch, a sugar, a microcrystalline cellulose, a hydroxy propyl methyl cellulose, a hydroxy propyl cellulose, or a hydroxy ethyl cellulose, or any combination thereof; d) 25 wt % to 50 wt % of a filler by the weight of the pharmaceutical composition; wherein the filler is selected from a microcrystalline cellulose, a lactose, a sorbitol, a cellulose, a calcium phosphate, a starch, or a sugar, or any combination thereof; and e) 0.5 wt % to 3 wt % of a lubricant by the weight of the composition, wherein the lubricant is selected from a metal stearate and/or a metal stearyl fumarate. Specific examples of the fillers, disintegrant agents, binders, and lubricants are as described above.

In yet another further specific embodiment, the pharmaceutical compositions of the invention comprise: a) 35 wt % to 75 wt % of Form A of HCl salt of Compound (1)•½ H₂O by the weight of the pharmaceutical composition; b) 3 wt % to 7 wt % of a croscarmellose by the weight of the pharmaceutical composition; c) 0.5 wt % to 2 wt % a polyvinyl pyrrolidone by the weight of the pharmaceutical composition; d) 25 wt % to 50 wt % of a filler by the weight of the pharmaceutical composition; wherein the filler includes a microcrystalline cellulose and a lactose; and e) 0.5 wt % to 3 wt % of a metal stearyl fumarate by the weight of the composition. Specific examples of the fillers, disintegrant agents, binders, and lubricants are as described above.

In yet another further specific embodiment, the pharmaceutical compositions of the invention comprise: a) 35 wt % to 75 wt % of Form A of HCl salt of Compound (1)•½ H₂O by the weight of the pharmaceutical composition; b) 3 wt % to 7 wt % of a croscarmellose by the weight of the pharmaceutical composition; c) 0.5 wt % to 2 wt % of a polyvinyl pyrrolidone by the weight of the pharmaceutical composition; d) 25 wt % to 50 wt % of a filler by the weight of the pharmaceutical composition; wherein the filler includes a microcrystalline cellulose and a lactose; and e) 0.5 wt % to 3 wt % of sodium stearyl fumarate by the weight of the composition. Specific examples of the fillers, disintegrant agents, binders, and lubricants are as described above.

In yet another further specific embodiment, the pharmaceutical compositions of the invention comprise: a) 35 wt % to 65 wt % of Form A of HCl salt of Compound (1)•½ H₂O by the weight of the pharmaceutical composition; b) 3 wt % to 7 wt % of croscarmellose sodium by the weight of the pharmaceutical composition; c) 0.5 wt % to 2 wt % of a polyvinyl pyrrolidone having an average molecular weight of 3,000 to 5,000 by the weight of the pharmaceutical composition; d) 30 wt % to 40 wt % of a microcrystalline cellulose by the weight of the pharmaceutical composition; e) 5 wt % to 10 wt % of lactose monohydrate by the weight of the pharmaceutical composition; and f) 1 wt % to 3 wt % of sodium stearyl fumarate by the weight of the composition.

In yet another further specific embodiment, the pharmaceutical compositions of the invention comprise: a) 35 wt % to 65 wt % of Form A of HCl salt of Compound (1)•½ H₂O by the weight of the pharmaceutical composition; b) 0.5 wt % to 2 wt % of colloidal silica by the weight of the pharmaceutical composition; c) 5 wt % to 30 wt %, 10 wt % to 25 wt % of silicified microcrystalline celluloses by the weight of the pharmaceutical composition; d) 0.5 wt % to 20 wt %, 5 wt % to 10 wt % of a microcrystalline cellulose by the weight of the pharmaceutical composition; e) 1 wt % to 7 wt %, 1.5 wt % to 5 wt % starch (e.g., pregelatinized starch) by the weight of the composition; f) 3 wt % to 7 wt % of crospovidone by the weight of the pharmaceutical composition; and g) 1 wt % to 7 wt %, 1.5 wt % to 5 wt % of sodium stearyl fumarate by the weight of the composition.

In another aspect, the pharmaceutical compositions of the invention are intravenous (IV) formulations that comprise Compound (1) in water and 0.01 M to 0.1 M of a pharmaceutically acceptable pH modifier, such as a pH buffering agent. Typically, the pharmaceutical compositions include: 1 mg/mL to 20 mg/mL of Compound (1) in solution. More typically, the pharmaceutical compositions include: 1 mg/mL to 10 mg/mL of Compound (1) or 1 mg/mL to 5 mg/mL of Compound (1), such as 2 mg/mL of Compound (1). In one embodiment, a HCl salt of Compound (1)•xH₂O (wherein x is 0 to 3) are employed as a source of Compound (1) of the IV formulations. Without intending to be bound to a particular theory, a HCl salt of Compound (1)•xH₂O exists as Compound (1) in solution. Typical examples of polymorphic forms of HCl salt of Compound (1)•xH₂O are as described above. In one specific embodiment, Form A, Form D, or Form F of HCl salt of Compound (1)•xH₂O is employed. In another specific embodiment, Form A of HCl salt of Compound (1)•½ H₂O is employed.

Typical examples of pH modifiers include NaOH, KOH, NH₄OH, HCl, and buffering agents. Typical examples of buffering agents include carbonates, bicarbonates, monobasic phosphates, dibasic phosphates, and acetates. Specific example of buffering agents includes phosphate buffering agents, such as monosodium phosphate and disodium phosphate. In one specific embodiment, a mixture of monosodium phosphate and disodium phosphate is employed as the buffering agent.

In one embodiment, the IV formulations further comprise 1 wt % to 20 wt % of a complexing agent by weight of the IV formulations. Typical complexing agents include cyclodextrins (e.g., an alpha cyclodextrin, a beta cyclodextrin, a gamma cyclodextrin, a hydroxypropyl-beta-cyclodextrin, a sulfo-butylether-beta-cyclodextrin, and a polyanionic beta-cyclodextrin), polysorbates (e.g., Tween® 80), and castor oils (e.g., Cremophor® series). Specific examples of cyclodextrins include an alpha cyclodextrin (e.g., Cavamax® W6), a beta cyclodextrin (e.g., Cavamax® W7), a gamma cyclodextrin (e.g., Cavamax® W8), a hydroxypropyl-beta-cyclodextrin (e.g., Cavasol® W7, Cavitron® W7), a sulfo-butylether-beta-cyclodextrin, and a polyanionic beta-cyclodextrin (e.g., Captisol®). A specific example of polysorbate includes a polyoxyethylene (20) sorbitan monoleate (e.g., Tween® 80). Specific examples of castor oils include a polyoxy 40 hydrogenated castor oil (e.g., Cremophor® RH 40), a polyoxy 35 castor oil (e.g., Cremophor® EL). In one specific embodiment, the complexing agents are selected from a polyoxy 40 hydrogenated castor oil, a polyoxy 35 castor oil, a polyanionic beta-cyclodextrin, or a hydroxypropyl-beta-cyclodextrin, or any combination thereof.

In some embodiments, the IV formulations further comprise a dextrose and/or a manitol as tonicity modifiers.

In some embodiments, the IV formulations further comprise a buffer.

In some embodiments, the pharmaceutical compositions of the invention further comprise a colorant, such as Opadry II white.

In some embodiments, the pharmaceutical compositions of the invention are in solid dosage forms, specifically in tablet forms.

In another aspect, the present invention covers methods of preparing the pharmaceutical compositions described above. In one embodiment, the methods comprise providing a mixture of Compound (1) that includes: a) 5 wt % to 95 wt % of a HCl salt of Compound (1)•xH₂O (wherein x is from 0 to 3 (e.g., 0.5)) by the weight of the pharmaceutical composition; and b) 5 wt % to 95 wt % of a filler by the weight of the pharmaceutical composition. In another embodiment, the methods comprise providing a mixture of Compound (1) that includes: a) 20 wt % to 80 wt % of a HCl salt of Compound (1)•xH₂O (wherein x is from 0 to 3) by the weight of the pharmaceutical composition; and b) 20 wt % to 80 wt % of a filler by the weight of the pharmaceutical composition. In one specific embodiment, the step of providing the mixture of Compound (1) includes: to provide granules of Compound (1), mixing i) 60 wt % to 90 wt % of HCl salt of Compound (1)•xH₂O by the weight of the granules of Compound (1) and ii) an intra-granular excipient that includes 10 wt % to 40 wt % of the filler by the weight of the granules of Compound (1); and mixing the granules of Compound (1) with an extra-granular excipient that includes 15 wt % to 40 wt % of the filler by the weight of the pharmaceutical composition.

In another specific embodiment, the pharmaceutical compositions of the invention further includes a binder, a disintegrant, and a lubricant, and the step of providing the mixture of Compound (1) includes: to provide granules of Compound (1), mixing i) 70 wt % to 85 wt % of HCl salt of Compound (1)•xH₂O by the weight of the granules of Compound (1) and ii) an intra-granular excipient that includes 14 wt % to 25 wt % of the filler by the weight of the granules of Compound (1) and 1 wt % to 5 wt % of the disintegrant agent by the weight of the granules of Compound (1); and mixing the granules of Compound (1) with an extra-granular excipient that includes 15 wt % to 40 wt % of the filler by the weight of the pharmaceutical composition, 0.5 wt % to 5 wt % of the disintegrant agent by the weight of the pharmaceutical composition, and 0.5 wt % to 5 wt % of the lubricant by the weight of the pharmaceutical composition.

In yet another specific embodiment, the step of providing the mixture of Compound (1) includes: providing a binder solution that includes water and 0.5 wt % to 5 wt % of the binder by the weight of the granules; providing an intra-granulation composition to provide granules of Compound (1), the intra-granulation composition including: i) 70 wt % to 85 wt % of HCl salt of Compound (1)•xH₂O by the weight of the granules of Compound (1) and ii) an intra-granular excipient that includes 14 wt % to 25 wt % of the filler by the weight of the granules of Compound (1) and 1 wt % to 5 wt % of the disintegrant agent by the weight of the granules of Compound (1); mixing the binder solution and the pre-granulation composition to form the granules of Compound (1); and mixing the granules of Compound (1) with an extra-granular excipient that includes 15 wt % to 40 wt % of the filler by the weight of the pharmaceutical composition, 0.5 wt % to 5 wt % of the disintegrant agent by the weight of the pharmaceutical composition, and 0.5 wt % to 5 wt % of the lubricant by the weight of the pharmaceutical composition.

The granules of Compound (1) can be made in any suitable way known in the art, such as twin screw wet granulation or high shear wet granulation. In one embodiment, twin screw wet granulation is employed for the preparation of granules of Compound (1). In a specific embodiment, the step of mixing the binder solution and the pre-granulation composition includes: i) feeding the pre-granulation composition into a twin screw extruder; and ii) introducing the binder solution into the twin screw extruder. In a further specific embodiment, the binder solution includes water in a range of 30 wt % to 50wt % of the weight of the intra-granulation composition.

The granules of Compound (1) are milled and the milled granules are mixed with an extra-granular composition that includes a filler and other ingredients as desired (e.g., disintegrant and/or a lubricant). In some embodiments, 60 wt % to 80 wt % of the milled granules of Compound (1) are mixed with 10 wt % to 30 wt % of filler, and optionally further with 1 wt % to 15 wt % of disintegrant and/or 0.25 wt % to 5 wt % of lubricant, by the total combined weight.

For tablet compositions of the invention, the methods further comprise film coating the tablet compositions. Typical film coating materials include one or more colorants, such as Opadry II white.

Methods of preparing the IV formulations described above are also provided here. Typically, the methods comprise mixing: a) a HCl salt of Compound (1)•xH₂O (wherein x is 0-3); and b) 0.01 M to 0.1 M of a pH modifier to from 1 mg/mL to 20 mg/mL of compound (1) in water. In some embodiments, 1 mg/mL to 10 mg/mL of compound (1) is formed. As described above for the IV formulations, other ingredients, such as complexing agents and/or modifiers may also be mixed with the HCl salt of Compound (1)•xH₂O and pH modifier.

Examples, including specific examples, of the HCl salts of Compound (1)•xH₂O, fillers, disintegrant agents, binders, and lubricants, pH modifiers, complexing agents, and modifiers which can be employed for the methods of preparing pharmaceutical compositions are each and independently as described above for the pharmaceutical compositions of the invention.

The pharmaceutical compositions of the invention are pharmaceutically acceptable. As used herein, “pharmaceutically acceptable” means being inert without unduly inhibiting the biological activity of the active compound(s) (e.g. HCl salts of Compound (1)•xH₂O), and biocompatible (e.g., non-toxic, non-inflammatory, non-immunogenic or devoid of other undesired reactions or side-effects upon the administration to a subject).

The pharmaceutical compositions of the invention may further include one or more pharmaceutically acceptable carriers other than those described above. The pharmaceutically acceptable carriers should be biocompatible. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as phosphates or glycine,), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Combination Therapies

An effective amount can be achieved in the method or pharmaceutical composition of the invention employing a compound of the invention (including a pharmaceutically acceptable salt or solvate (e.g., hydrate)) alone or in combination with an additional suitable therapeutic agent, for example, an antiviral agent or a vaccine (such as oseltamivir (Tamiflu®) or zanamivir (Rolenza®)). When a “combination therapy” is employed, an effective amount can be achieved using a first amount of a compound or pharmaceutical composition of the invention and a second amount of an additional suitable therapeutic agent. In some embodiments of the present invention, the additional therapeutic agent can be a neuraminidase inhibitor, such as oseltamivir (Tamiflu®) or zanamivir (Rolenza®).

In another embodiment, any pharmaceutical composition described herein can also include one or more additional agents, such as a neuraminidase inhibitor like oseltamivir (e.g., oseltamivir phosphate) (Tamiflu®) or zanamivir (Rolenza®).

In another embodiment, a compound of the invention and the additional therapeutic agent, are each administered in an effective amount (i.e., each in an amount which would be therapeutically effective if administered alone). In another embodiment, a compound of the invention and the additional therapeutic agent, are each administered in an amount which alone does not provide a therapeutic effect (a sub-therapeutic dose). In yet another embodiment, a compound of the invention can be administered in an effective amount, while the additional therapeutic agent is administered in a sub-therapeutic dose. In still another embodiment, a compound of the invention can be administered in a sub-therapeutic dose, while the additional therapeutic agent, for example, a suitable cancer-therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the compounds of the co-administration in an essentially simultaneous manner, such as in a single pharmaceutical composition, for example, capsule or tablet having a fixed ratio of first and second amounts, or in multiple, separate capsules or tablets for each. In addition, such co-administration also encompasses use of each compound in a sequential manner in either order.

In one embodiment, the present invention is directed to methods for inhibiting influenza viruses replication in biological samples or patients, or for treating or preventing influenza virus infections in patients using the compounds described herein. Accordingly, pharmaceutical compositions of the invention also include those comprising an inhibitor of influenza virus replication in combination with an anti-viral compound exhibiting anti-influenza virus activity.

Methods of use of the compounds described herein and compositions of the invention also include combination of chemotherapy with a compound or composition of the invention, or with a combination of a compound or composition of this invention with another anti-viral agent and vaccination with a Flu vaccine.

When co-administration involves the separate administration of the first amount of a compound of the invention and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, a compound of the invention and the second therapeutic agent can be administered in any order within 24 hours of each other, within 16 hours of each other, within 8 hours of each other, within 4 hours of each other, within 1 hour of each other or within 30 minutes of each other.

More, specifically, a first therapy (e.g., a prophylactic or therapeutic agent such as a compound of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-cancer agent) to a subject.

It is understood that the method of co-administration of a first amount of a compound of the invention and a second amount of an additional therapeutic agent can result in an enhanced or synergistic therapeutic effect, wherein the combined effect is greater than the additive effect that would result from separate administration of the first amount of a compound of the invention and the second amount of an additional therapeutic agent.

As used herein, the term “synergistic” refers to a combination of a compound of the invention and another therapy (e.g., a prophylactic or therapeutic agent), which is more effective than the additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using the compounds of the present invention is in combination with a Flu vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T.C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

Specific examples that can be co-administered with a compound described herein include neuraminidase inhibitors, such as oseltamivir (Tamiflu®) and Zanamivir (Rlenza®), viral ion channel (M2 protein) blockers, such as amantadine (Symmetrel®) and rimantadine (Flumadine®), and antiviral drugs described in WO 2003/015798, including T-705 under development by Toyama Chemical of Japan. (See also Ruruta et al., Antiviral Research, 82: 95-102 (2009), “T-705 (flavipiravir) and related compounds: Novel broad-spectrum inhibitors of RNA viral infections”). In some embodiments, the compounds described herein can be co-administered with a traditional influenza vaccine. In some embodiments, the compounds described herein can be co-administered with zanamivir. In some embodiments, the compounds described herein can be co-administered with oseltamivir. In some embodiments, the compounds described herein can be co-administered with flavipiravir (T-705). In some embodiments, the compounds described herein can be co-administered with amantadine or rimantadine. Oseltamivir can be administered in a dosage regimen according to its label. In some specific embodiments, it is administered 75 mg twice a day, or 150 mg once a day.

Uses of the Pharmaceutical Compositions

One aspect of the present invention is generally related to the use of the pharmaceutically acceptable compositions described above, useful for inhibiting the replication of influenza viruses in a biological sample or in a patient, for reducing the amount of influenza viruses (reducing viral titer) in a biological sample or in a patient, and for treating influenza in a patient. Hereinafter unless specifically indicated otherwise, the various solid forms (e.g., polymorphs of HCl salts of Compound (1) or pharmaceutically acceptable salts thereof) described above are also referred to generally compounds.

In one embodiment, the present invention is generally related to the use of the compounds disclosed herein (e.g., in pharmaceutically acceptable compositions) for any of the uses specified above.

In yet another embodiment, the compounds disclosed herein can be used to reduce viral titre in a biological sample (e.g. an infected cell culture) or in humans (e.g. lung viral titre in a patient).

The terms “influenza virus mediated condition”, “influenza infection”, or “Influenza”, as used herein, are used interchangeable to mean the disease caused by an infection with an influenza virus.

Influenza is an infectious disease that affects birds and mammals caused by influenza viruses. Influenza viruses are RNA viruses of the family Orthomyxoviridae, which comprises five genera: Influenza virus A, Influenza virus B, Influenza virus C, ISA virus and Thogoto virus. Influenza virus A genus has one species, influenza A virus which can be subdivided into different serotypes based on the antibody response to these viruses: H1N1, H2N2, H3N2, H5N1, H7N7, H1N2, H9N2, H7N2, H7N3 and H1ON7. Additional examples of influenza A virus include H3N8 and H7N9. Influenza virus B genus has one species, influenza B virus. Influenza B almost exclusively infects humans and is less common than influenza A. Influenza virus C genus has one species, Influenza virus C virus, which infects humans and pigs and can cause severe illness and local epidemics. However, Influenza virus C is less common than the other types and usually seems to cause mild disease in children.

In some embodiments of the invention, influenza or influenza viruses are associated with Influenza virus A or B. In some embodiments of the invention, influenza or influenza viruses are associated with Influenza virus A. In some specific embodiments of the invention, Influenza virus A is H1N1, H2N2, H3N2 or H5N1. In some specific embodiments of the invention, Influenza virus A is H1N1, H3N2, H3N8, H5N1, and H7N9. In some specific embodiments of the invention, Influenza virus A is H1N1, H3N2, H3N8, and H5N1.

In humans, common symptoms of influenza are chills, fever, pharyngitis, muscle pains, severe headache, coughing, weakness, and general discomfort. In more serious cases, influenza causes pneumonia, which can be fatal, particularly in young children and the elderly. Although it is often confused with the common cold, influenza is a much more severe disease and is caused by a different type of virus. Influenza can produce nausea and vomiting, especially in children, but these symptoms are more characteristic of the unrelated gastroenteritis, which is sometimes called “stomach flu” or “24-hour flu”.

Symptoms of influenza can start quite suddenly one to two days after infection. Usually the first symptoms are chills or a chilly sensation, but fever is also common early in the infection, with body temperatures ranging from 38-39° C. (approximately 100-103° F.). Many people are so ill that they are confined to bed for several days, with aches and pains throughout their bodies, which are worse in their backs and legs. Symptoms of influenza may include: body aches, especially joints and throat, extreme coldness and fever, fatigue, headache, irritated watering eyes, reddened eyes, skin (especially face), mouth, throat and nose, abdominal pain (in children with influenza B). Symptoms of influenza are non-specific, overlapping with many pathogens (“influenza-like illness). Usually, laboratory data is needed in order to confirm the diagnosis.

The terms, “disease”, “disorder”, and “condition” may be used interchangeably here to refer to an influenza virus mediated medical or pathological condition.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

The term “biological sample”, as used herein, includes, without limitation, cell cultures or extracts thereof; biopsied material obtained from a mammal or extracts thereof; blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the term “inhibition of the replication of influenza viruses” includes both the reduction in the amount of virus replication (e.g. the reduction by at least 10%) and the complete arrest of virus replication (i.e., 100% reduction in the amount of virus replication). In some embodiments, the replication of influenza viruses are inhibited by at least 50%, at least 65%, at least 75%, at least 85%, at least 90%, or at least 95%.

Influenza virus replication can be measured by any suitable method known in the art. For example, influenza viral titre in a biological sample (e.g. an infected cell culture) or in humans (e.g. lung viral titre in a patient) can be measured. More specifically, for cell based assays, in each case cells are cultured in vitro, virus is added to the culture in the presence or absence of a test agent, and after a suitable length of time a virus-dependent endpoint is evaluated. For typical assays, the Madin-Darby canine kidney cells (MDCK) and the standard tissue culture adapted influenza strain, A/Puerto Rico/8/34 can be used. A first type of cell assay that can be used in the invention depends on death of the infected target cells, a process called cytopathic effect (CPE), where virus infection causes exhaustion of the cell resources and eventual lysis of the cell. In the first type of cell assay, a low fraction of cells in the wells of a microtiter plate are infected (typically 1/10 to 1/1000), the virus is allowed to go through several rounds of replication over 48-72 hours, then the amount of cell death is measured using a decrease in cellular ATP content compared to uninfected controls. A second type of cell assay that can be employed in the invention depends on the multiplication of virus-specific RNA molecules in the infected cells, with RNA levels being directly measured using the branched-chain DNA hybridization method (bDNA). In the second type of cell assay, a low number of cells are initially infected in wells of a microtiter plate, the virus is allowed to replicate in the infected cells and spread to additional rounds of cells, then the cells are lysed and viral RNA content is measured. This assay is stopped early, usually after 18-36 hours, while all the target cells are still viable. Viral RNA is quantitated by hybridization to specific oligonucleotide probes fixed to wells of an assay plate, then amplification of the signal by hybridization with additional probes linked to a reporter enzyme.

As used herein a “viral titer (or titre)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. A specific example is viral titer. To determine the titer, several dilutions will be prepared, such as 10-1, 10-2, 10-3, 10-4, 10-5, 10-6, 10-7, 10-8, or the like. The lowest concentration of virus that still infects cells is the viral titer.

As used herein, the terms “treat”, “treatment” and “treating” refer to both therapeutic and prophylactic treatments. For example, therapeutic treatments includes the reduction or amelioration of the progression, severity and/or duration of influenza viruses mediated conditions, or the amelioration of one or more symptoms (specifically, one or more discernible symptoms) of influenza viruses mediated conditions, resulting from the administration of one or more therapies (e.g., one or more therapeutic agents such as a compound or composition of the invention). In specific embodiments, the therapeutic treatment includes the amelioration of at least one measurable physical parameter of an influenza virus mediated condition. In other embodiments the therapeutic treatment includes the inhibition of the progression of an influenza virus mediated condition, either physically by, e.g., stabilization of a discernible symptom, physiologically by, e.g., stabilization of a physical parameter, or both. In other embodiments the therapeutic treatment includes the reduction or stabilization of influenza viruses mediated infections. Antiviral drugs can be used in the community setting to treat people who already have influenza to reduce the severity of symptoms and reduce the number of days that they are sick.

The term “chemotherapy” refers to the use of medications, e.g. small molecule drugs (rather than “vaccines”) for treating a disorder or disease.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious influenza complications live in close contact with each other (e.g. in a hospital ward, daycare center, prison, nursing home, etc.). It also includes the use among populations who require protection from the influenza but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the influenza or not considered at high risk for complications, in order to reduce the chances of getting infected with the influenza and passing it on to a high-risk person in close contact with him (for instance, healthcare workers, nursing home workers, etc.).

According to the US CDC, an influenza “outbreak” is defined as a sudden increase of acute febrile respiratory illness (AFRI) occurring within a 48 to 72 hour period, in a group of people who are in close proximity to each other (e.g. in the same area of an assisted living facility, in the same household, etc.) over the normal background rate or when any subject in the population being analyzed tests positive for influenza. One case of confirmed influenza by any testing method is considered an outbreak.

A “cluster” is defined as a group of three or more cases of AFRI occurring within a 48 to 72 hour period, in a group of people who are in close proximity to each other (e.g. in the same area of an assisted living facility, in the same household, etc.).

As used herein, the “index case”, “primary case” or “patient zero” is the initial patient in the population sample of an epidemiological investigation. When used in general to refer to such patients in epidemiological investigations, the term is not capitalized. When the term is used to refer to a specific person in place of that person's name within a report on a specific investigation, the term is capitalized as Patient Zero. Often scientists search for the index case to determine how the disease spread and what reservoir holds the disease in between outbreaks. Note that the index case is the first patient that indicates the existence of an outbreak. Earlier cases may be found and are labeled primary, secondary, tertiary, and the like.

In one embodiment, the methods of the invention are a preventative or “pre-emptive” measure to a patient, specifically a human, having a predisposition to complications resulting from infection by an influenza virus. The term “pre-emptive” as used herein as for example in pre-emptive use, “pre-emptively”, etc., is the prophylactic use in situations in which an “index case” or an “outbreak” has been confirmed, in order to prevent the spread of infection in the rest of the community or population group.

In another embodiment, the methods of the invention are applied as a “pre-emptive” measure to members of a community or population group, specifically humans, in order to prevent the spread of infection.

As used herein, an “effective amount” refers to an amount sufficient to elicit the desired biological response. In the present invention the desired biological response is to inhibit the replication of influenza virus, to reduce the amount of influenza viruses or to reduce or ameliorate the severity, duration, progression, or onset of an influenza virus infection, prevent the advancement of an influenza viruses infection, prevent the recurrence, development, onset or progression of a symptom associated with an influenza virus infection, or enhance or improve the prophylactic or therapeutic effect(s) of another therapy used against influenza infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other antiviral agents, e.g., when co-administered with an anti-influenza medication, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, the compounds disclosed herein can be administered to a subject in a dosage range from between approximately 0.01 to 100 mg/kg body weight/day for therapeutic or prophylactic treatment.

Generally, dosage regimens can be selected in accordance with a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the renal and hepatic function of the subject; and the particular compound or salt thereof employed, the duration of the treatment; drugs used in combination or coincidental with the specific compound employed, and like factors well known in the medical arts. The skilled artisan can readily determine and prescribe the effective amount of the compounds described herein required to treat, to prevent, inhibit (fully or partially) or arrest the progress of the disease.

Dosages of the compounds described herein can range from 0.01 to 100 mg/kg body weight/day, 0.01 to 50 mg/kg body weight/day, 0.1 to 50 mg/kg body weight/day, or 1 to 25 mg/kg body weight/day. It is understood that the total amount per day can be administered in a single dose or can be administered in multiple dosing, such as twice a day (e.g., every 12 hours or with 4 to 10 hours interval), three times a day (e.g., every 8 hours or with 4 to 10 hours interval), or four times a day (e.g., every 6 hours or with 4 to 10 hours interval).

In some embodiments, dosages of the compounds described herein (e.g., Compound (1) and its pharmaceutically acceptable salts thereof, including the various solid forms (e.g., Form A of HCl salt of Compound (1)•½ H₂O, Form F of HCl salt of Compound (1)•3H₂O, Form D of HCl salt of Compound (1)) are in a range of 100 mg to 1,600 mg, such as 400 mg to 1,600 mg or 400 mg to 1,200 mg. Each dose can be taken once a day (QD), twice per day (e.g., every 12 hours or with 4 to 10 hours interval (BID)), or three times per day (e.g., q8h or with 4 to 10 hours interval (TID)). It is noted that any combinations of QD, BID, and TID can be employed, as desired, such as BID on day 1, followed by QD thereafter, or, when a loading dosage is employed on day 1, BID on day 2, followed by QD thereafter.

In one specific embodiment, dosages of the compounds described herein are 400 mg to 1,600 mg, 400 mg to 1,200 mg, or 600 mg to 1,200 mg once a day. In another specific embodiment, dosages of the compounds described herein are 400 mg to 1,600 mg, 400 mg to 1,200 mg, 300 mg to 900 mg or 400 mg to 600 mg twice a day. In yet another specific embodiment, dosages of the compounds described herein are 400 mg to 1,000 mg once a day. In yet another specific embodiment, dosages of the compounds described herein are 600 mg to 1,000 mg once a day. In yet another specific embodiment, dosages of the compounds described herein are 600 mg to 800 mg once a day. In yet another specific embodiment, dosages of the compounds described herein are 400 mg to 800 mg twice a day (e.g., 400 mg to 800 mg every 12 hours or with 4 to 10 hours interval). In yet another specific embodiment, dosages of the compounds described herein are 400 mg to 600 mg twice a day.

In some embodiments, a loading dosage regimen is employed. In one specific embodiment, a loading dose of 400 mg to 1,600 mg is employed on day 1 of treatment. In another specific embodiment, a loading dose of 600 mg to 1,600 mg is employed on day 1 of treatment. In another specific embodiment, a loading dose of 800 mg to 1,600 mg is employed on day 1 of treatment. In yet another specific embodiment, a loading dose of 900 mg to 1,600 mg is employed on day 1 of treatment. In yet another specific embodiment, a loading dose of 900 mg to 1,200 mg is employed on day 1 of treatment. In yet another specific embodiment, a loading dose of 900 mg is employed on day 1 of treatment. In yet another specific embodiment, a loading dose of 1,000 mg is employed on day 1 of treatment. In yet another specific embodiment, a loading dose of 1,200 mg is employed on day 1 of treatment.

In one specific embodiment, the dosage regimen of the compounds described herein employs a loading dosage of 600 mg to 1,600 mg on day 1 and with a regular dosage of 300 mg to 1,200 mg for the rest of the treatment duration. Each regular dose can be taken once a day, twice a day, or three times a day, or any combination thereof. In a further specific embodiment, a loading dosage of 900 mg to 1,600 mg, such as 900 mg, 1,200 mg, or 1,600 mg, is employed. In another further specific embodiment, a loading dosage of 900 mg to 1,200 mg, such as 900 mg or 1,200 mg, is employed. In yet another further specific embodiment, a regular dosage of 400 mg to 1,200 mg, such as 400 mg, 600 mg, or 800 mg, is employed for the rest of the treatment duration. In yet another further specific embodiment, a regular dosage of 400 mg to 1,000 mg for the rest of the treatment duration. In yet another further specific embodiment, a regular dosage of 400 mg to 800 mg is employed for the rest of the treatment duration. In yet another further specific embodiment, a regular dosage of 300 mg to 900 mg twice a day is employed. In yet another further specific embodiment, a regular dosage of 600 mg to 1,200 mg once a day is employed. In yet another further specific embodiment, a regular dosage of 600 mg twice a day on day 2, followed by 600 mg once a day for the rest of the treatment duration.

For therapeutic treatment, the compounds described herein can be administered to a patient within, for example, 48 hours (or within 40 hours, or less than 2 days, or less than 1.5 days, or within 24 hours) of onset of symptoms (e.g., nasal congestion, sore throat, cough, aches, fatigue, headaches, and chills/sweats). Alternatively, for therapeutic treatment, the compounds described herein can be administered to a patient within, for example, 96 hours of onset of symptoms. In certain embodiments, administration is thus first effected with 48 to 96 hours of onset of influenza symptoms. It is preferred that administration be first effected within about 60 to about 96 hours of onset of symptoms, preferably within about 72 to about 96 hours, more preferably within about 72 hours. It is therefore intended that in certain embodiments of the present invention, administration will be first effected while the patient is hospitalized. In certain embodiments, administration is first effected after the patient's oxygen saturation level has fallen below 90%, 92%, 94%, 96% or 98% typically measured by pulse oximetry, and/or after the patient has been deemed to require administration of supplemental oxygen.

“Hospitalized” refers to patients or subjects requiring hospitalization to treat influenza infection and/or to treat complications of influenza infection (e.g., radiological signs of lower respiratory tract disease, septic shock, central nervous system [CNS] involvement, myositis, rhabdomyolysis, acute exacerbation of chronic kidney disease, severe dehydration, myocarditis, pericarditis, ischemic heart disease, exacerbation of underlying chronic pulmonary disease, including asthma, chronic obstructive pulmonary disease [COPD], decompensation of previously controlled diabetes mellitus), including subjects admitted to the intensive care unit (ICU) and subjects admitted under “observation” status with an anticipated length of stay beyond 24 hours.

The therapeutic treatment can last for any suitable duration, for example, for 3 days, 4 days, 5 days, 7 days, 10 days, 14 days, etc. For prophylactic treatment during a community outbreak, the compounds described herein can be administered to a patient within, for example, 2 days of onset of symptoms in the index case, and can be continued for any suitable duration, for example, for 7 days, 10 days, 14 days, 20 days, 28 days, 35 days, 42 days, etc., up to the entire flu season. A flu season is an annually-recurring time period characterized by the prevalence of outbreaks of influenza. Influenza activity can sometimes be predicted and even tracked geographically. While the beginning of major flu activity in each season varies by location, in any specific location these minor epidemics usually take 3-4 weeks to peak and another 3-4 weeks to significantly diminish. Typically, Centers for Disease Control (CDC) collects, compiles and analyzes information on influenza activity year round in the United States and produces a weekly report from October through mid-May.

In one embodiment, the therapeutic treatment lasts for 1 day to an entire flu season. In one specific embodiment, the therapeutic treatment lasts for 3 days to 14 days. In another specific embodiment, the therapeutic treatment lasts for 5 days to 14 days. In another specific embodiment, the therapeutic treatment lasts for 3 days to 10 days. In yet another specific embodiment, the therapeutic treatment lasts for 4 days to 10 days. In yet another specific embodiment, the therapeutic treatment lasts for 5 days to 10 days. In yet another specific embodiment, the therapeutic treatment lasts for 4 days to 7 days (e.g., 4 days, 5 days, 6 days, or 7 days). In yet another specific embodiment, the therapeutic treatment lasts for 5 days to 7 days (e.g., 5 days, 6 days, or 7 days). In one specific embodiment, the prophylactic treatment lasts up to the entire flu season.

In one specific embodiment, the compounds described herein are administered to a patient for 3 days to 14 days (e.g., 5 days to 14 days) with a loading dosage of 900 mg to 1,600 mg on day 1 and with a regular dosage of 300 mg to 1,200 mg for the rest of the treatment duration. In another specific embodiment, the compounds described herein are administered to a patient for 3 days to 14 days (e.g., 5 days to 14 days) with a loading dosage of 900 mg to 1,200 mg on day 1 and with a regular dosage of 400 mg to 1,000 mg for the rest of the treatment duration. In yet another specific embodiment, the compounds described herein are administered to a patient for 3 days to 14 days (e.g., 5 days to 14 days) with a loading dosage of 900 mg to 1,200 mg on day 1 and with a regular dosage of 400 mg to 800 mg for the rest of the treatment duration. In yet another specific embodiment, the compounds described herein are administered to a patient for 3 days to 14 days (e.g., 5 days to 14 days) with a loading dosage of 900 mg to 1,200 mg on day 1 and with a regular dosage of 400 mg to 800 mg for the rest of the treatment duration. Each dose can be taken once a day, twice a day, or three times a day, or any combination thereof.

In one specific embodiment, the compounds described herein are administered to a patient for 3 days to 14 days with a loading dosage of 900 mg to 1,600 mg on day 1 and with a regular dosage of 600 mg to 1,000 mg once a day for the rest of the treatment duration. In another specific embodiment, the compounds described herein are administered to a patient for 3 days to 14 days with a loading dosage of 900 mg to 1,200 mg on day 1 and with a regular dosage of 600 mg to 800 mg (e.g., 600 mg, 650 mg, 700 mg, 750 mg, or 800 mg) once a day for the rest of the treatment duration. In some embodiments, the treatment duration is for 4 days to 10 days, 5 days to 10 days, or 5 days to 7 days.

In one specific embodiment, the compounds described herein are administered to a patient for 3 days to 14 days with a loading dosage of 900 mg to 1,600 mg on day 1 and with a regular dosage of 400 mg to 800 mg twice a day for the rest of the treatment duration. In another specific embodiment, the compounds described herein are administered to a patient for 3 days to 14 days with a loading dosage of 900 mg to 1,200 mg on day 1 and with a regular dosage of 400 mg to 600 mg (e.g., 400 mg, 450 mg, 500 mg, 550 mg, or 600 mg) twice a day for the rest of the treatment duration. In some embodiments, the duration is for 4 days to 10 days, 5 days to 10 days, or 5 days to 7 days.

In one specific embodiment, the compounds described herein are administered to a patient for 4 days or 5 days with a loading dosage of 900 mg to 1,200 mg (e.g., 900 mg or 1,200 mg) on day 1 and with a regular dosage of 400 mg to 600 mg (e.g., 400 mg or 600 mg) twice a day for the rest of the treatment duration (e.g., days 2 through 4, or days 2 through 5). In another specific embodiment, the compounds described herein are administered to a patient for 4 days or 5 days with a loading dosage of 900 mg to 1,200 mg (e.g., 900 mg or 1,200 mg) on day 1 and with a regular dosage of 600 mg to 800 mg (e.g., 600 mg or 800 mg) once a day for the rest of the treatment duration.

In certain embodiments, the methods of the invention involve treating or reducing the severity of influenza virus infection comprising administering to a patient infected with influenza about 200 mg to about 800 mg, preferably about 600 mg, twice per day Compound (1) or a pharmaceutically acceptable salt thereof, preferably in combination with oseltamivir or a pharmaceutically acceptable salt thereof. In preferred embodiments, from about 50 mg to about 100 mg of oseltamivir is used, preferably about 75 mg.

In certain embodiments, the methods of the invention involve treating or reducing the severity of influenza virus infection comprising administering to a patient infected with influenza a pharmaceutical combination comprising from about 200 mg to about 800 mg of Compound (1) or a pharmaceutically acceptable salt thereof and from about 50 mg to about 100 mg of oseltamivir or a pharmaceutically acceptable salt thereof. A preferred combination includes about 600 mg of Compound (1) or a pharmaceutically acceptable salt thereof and about 75 mg of oseltamivir or a pharmaceutically acceptable salt thereof. The combination is administered at least once per day, preferably twice a day, and is first effected within 48 to 96 hours of onset of influenza symptoms in the patient, preferably within about 60 to about 96 hours, more preferably within about 72 to about 96 hours, still more preferably within about 72 hours. The treatment duration is 2, 3, 4, 5, 6, 7, 8, 9, 10, 11, 12, 13, 14 or 15 days.

Various types of administration methods can be employed in the invention, and are described in detail below under the section entitled “Administration Methods”.

Administration Methods

The compounds and pharmaceutically acceptable compositions described above can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compositions described herein may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tween®, Spans® and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., 1 to 4 or more times per day). When multiple daily doses are used, the unit dosage form can be the same or different for each dose.

III. EXAMPLES
Clinical Study

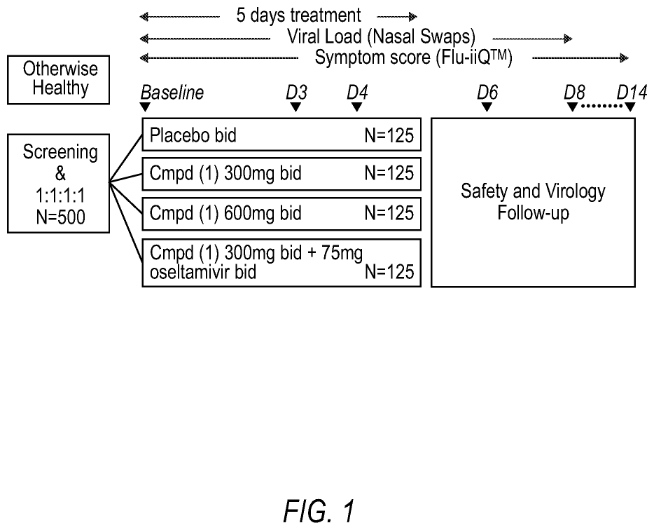

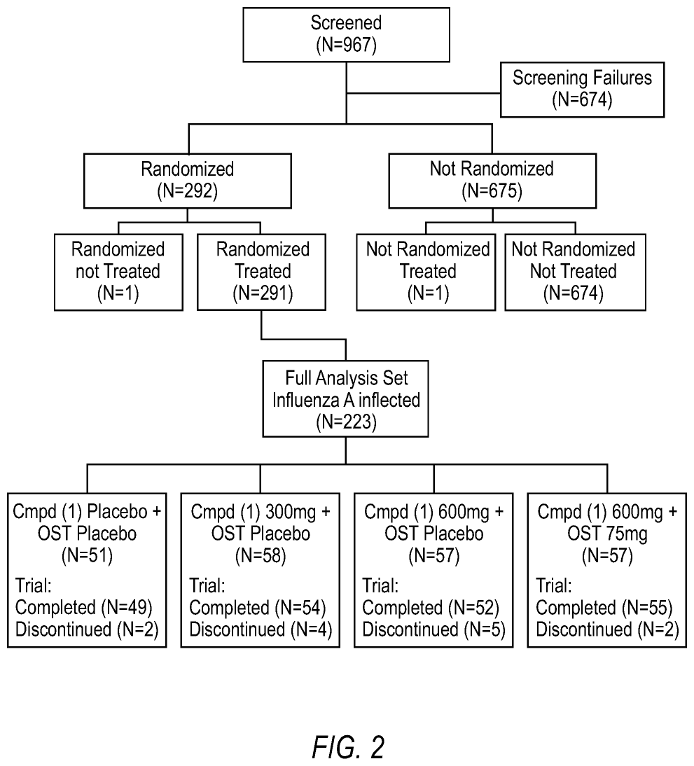

Referring to FIG. 1 and FIG. 2, a clinical study was undertaken to study the antiviral effects, as measured by viral load in nasal secretions in adult human patients with acute uncomplicated seasonal influenza A, following the administration of different dosages of Compound (1) and a combination therapy of Compound (1) and the neuraminidase inhibitor oseltamivir. This study followed a randomized, double-blind, placebo-controlled, parallel-group, multicenter design. The key design elements are described in FIG. 1, and a description of the randomization, treatment, completion, discontinuation, and screening failures is provided in the flow chart of FIG. 2.

Patients, 18-65 years in age, (N=500) presenting symptoms of acute influenza (i.e., oral temperature ≥38° C. (100.4° F.) within prior 24 hour period, presenting at least 1 respiratory symptom, presenting at least 1 systemic symptom, time of onset of flu-like symptoms no more than 48 hrs, and positive result for Rapid Influenza A Test at screening), were divided into 4 cohorts including a cohort (N=125) that was administered a placebo (bid), a cohort (N=125) that was administered 300 mg of Compound (1) (bid); a cohort (N=125) that was administered 600 mg of Compound (1) (bid); and a cohort (N=125) that was administered both 600 mg Compound (1) (bid) and 75 mg oseltamivir (bid).

Primary objective: Antiviral effect as measured by viral load in nasal secretions in adults with acute uncomplicated seasonal influenza A following administration of Compound (1).

Primary analysis set for efficacy: Full Analysis Set (FAS) included all randomly assigned subjects who received at least 1 dose of study drug and who had a confirmed influenza A infection. Confirmed infection was defined as a positive viral load result, either at baseline or at least at 2 post baseline time points.

Primary efficacy variable: Area under the curve (AUC) of the logio nasal viral load measured by quantitative reverse transcriptase polymerase chain reaction (qRT-PCR), from baseline to Day 8.

Key secondary efficacy variable: Time to resolution of influenza symptoms after initiation of study drug. Resolution of influenza symptoms was defined as the first Flu iiQ™ recording of a successive series of 3 recordings (over 4 scheduled successive analysis time points, 1 missing time point is allowed) in which all symptom scores for each of the 3 assessments were at most mild for all 7 primary influenza symptoms (cough, sore throat, headache, nasal stuffiness, feverishness or chills, muscle or joint pain, and fatigue).

Secondary efficacy variables: Duration of viral shedding defined as the time in days from the first dose of investigational drug until viral negativity (by qRT-PCR and median tissue culture infective dose); and time to resolution of fever defined as the time in hours from the first dose of investigational product until the time temperature equaled or became lower than 37.2° C. (99.0° F.).

Expected effect size and planned sample size: Sample size of 107 evaluable subjects per arm provided 80% power to detect a 1.67-day reduction (hazard ratio=1.5) in median time to symptom resolution in the active treatment arm compared to the placebo arm at a 5% level of significance, for a total of 428 evaluable subjects randomized and equally distributed over 4 treatment arms. Approximately 125 subjects per arm were needed to achieve approximately 107 influenza-infected subjects per arm, assuming a 10% dropout rate and an approximately 95% positivity rate for influenza A. This sample size was based on the primary endpoint of resolution of influenza symptoms in the original protocol.

TABLE 1
Median (95% CI)

| All placebo | Cmpd (1) 300 mg | Cmpd (1) 600 mg | Combination |
|---|---|---|---|
| 86.4 (68.7; | 99.0 (71.5; | 85.7 (55.3; | 70.4(61.8; |
| 117.3) | 150.6) | 114.9) | 82.5) |

Analysis set for safety: Safety Set included all subjects who received at least 1 dose of study drug.

Results summary:

After a formal interim analysis conducted at the end of the 2015/2016 Northern Hemisphere influenza season, the study was stopped for success as the primary endpoint was met. A summary of the final analysis follows.

In this double-blind, placebo-controlled, multicenter study, 293 subjects were enrolled, of which 223 subjects were randomized, treated, and had confirmed influenza A infection (Full Analysis Set (FAS)). In total, 292 subjects were treated (Safety Set), of which the majority were from the United States (78.8%), white (83.9%), and female (51.4%). The median age was 42 years, ranging from 18 to 65 years. No notable differences between treatment groups were observed.

Primary Efficacy Endpoint:

Antiviral activity was assessed using the area under the curve (AUC) of viral load (measured by quantitative reverse transcriptase polymerase chain reaction [qRT-PCR]) from Day 1 to Day 8 estimated on treatment group level using a mixed model for repeated measures. Differences between treatment groups were estimated adjusted for baseline viral load and stratum. These results showed an average reduction of AUC viral load versus placebo treatment of −3.6 (p=0.044), −4.5 (p=0.012), and −8.6 (p<0.001) day*log₁₀ copies/mL for the Compound (1) 300 mg twice daily (bid), Compound (1) 600 mg bid, and Compound (1) 600 mg bid+oseltamivir 75 mg bid treatment groups, respectively (see Table 2 below).

The average reduction in AUC viral load of Compound (1) 600 mg bid+oseltamivir 75 mg bid versus Compound (1) 600 mg bid treatment was -4.1 (p=0.017).

The primary analysis showed a statistically significant dose-response relationship: p-values of 0.009 and 0.010 (adjusted for multiplicity) for monotherapies combined versus placebo and a linear dose-response trend, respectively, as compared to the 1-sided type I error limit of 0.016. Adjustments were made for having multiple comparisons and performing an interim look to evaluate the primary study objective.

TABLE 2
Primary and Key Secondary Efficacy Results by
Treatment versus Placebo; Full Analysis Set.

| | Change in AUC Viral | | |
|---|---|---|---|
| | Load (by qRT-PCR) | | Time to Resolution |
| | Days 1 to 8 | | of Influenza Symptoms |
| | [day*log₁₀ copies/mL] | | Acceleration factor |
| Treatment Group | (95% CI) | | (95% CI) |
| Compound (1) 300 mg | −3.6 | (−7.1; −0.1) | 1.07 (0.76; 1.52) |
| bid | | | |
| Compound (1) 600 mg | −4.5 | (−8.0; −1.0) | 0.87 (0.62; 1.23) |
| bid | | | |
| Compound (1) 600 mg | −8.6 | (−12.0; −5.1) | 0.83 0.60; 1.16) |
| bid + oseltamivir | | | |
| 75 mg bid | | | |

Key Secondary Endpoint:

From the accelerated failure time model, it was estimated that there was an increase in time to resolution of the 7 primary influenza symptoms versus placebo of 7% for the Compound (1) 300 mg bid treatment group and a not statistically significant reduction in time to resolution of influenza symptoms versus placebo of 13% and 17% for the Compound (1) 600 mg bid and Compound (1) 600 mg bid+oseltamivir 75 mg bid treatment groups, respectively (see Table 2 above).

Other Secondary Endpoints:

From the accelerated failure time model on duration of viral load (by qRT-PCR), it was estimated that there was a reduction of time to viral negativity versus placebo treatment of 13%, 18%, and 31% for the Compound (1) 300 mg bid, Compound (1) 600 mg bid, and Compound (1) 600 mg bid+oseltamivir 75 mg bid treatment groups, respectively. The 95% confidence intervals for the acceleration factors were (0.74; 1.02), (0.70; 0.97), and (0.58; 0.81) for the Compound (1) 300 mg bid, Compound (1) 600 mg bid, and Compound (1) 600 mg bid+oseltamivir 75 mg bid treatment groups, respectively.

Safety:

The most common treatment-emergent adverse event (AE) was diarrhea: 6.9%, 6.8%, 27.0%, and 16.7% of subjects in the placebo, Compound (1) 300 mg bid, Compound (1) 600 mg bid, and Compound (1) 600 mg bid+oseltamivir 75 mg bid treatment groups, respectively.

No subjects died and for 2 subjects one treatment-emergent serious AE (SAE) was reported: an SAE of moderate increased alanine aminotransferase in the Compound (1) 600 mg bid treatment group (doubtfully related according to the investigator), and an SAE of severe thrombocytopenia in the placebo group (possibly related according to the investigator).

Conclusions:

Treatment with Compound (1) resulted in a statistically significant and dose-dependent decrease in AUC of viral load (by qRT-PCR) over 7 days from start of dosing. Further, Compound (1) in combination with oseltamivir resulted in a statistically significant lower AUC of viral load (by qRT-PCR) as compared to Compound (1) alone (600-mg dose).

Little separation in time to resolution of 7 primary influenza symptoms was found by the patient-reported outcome assessment, Flu-iiQ™. Given that the trial was finalized at the interim analysis for early success, the sample sizes per arm were relatively small, and clinical outcome comparisons had low power to show differences. The viral culture data were confirmatory of the qRT-PCR results but showed a shorter time to negativity compared to the qRT-PCR data.

Compound (1) was generally safe and well tolerated. A favorable safety profile was established. Increased incidences of diarrhea were reported; more common with 600 mg Compound (1) (as mono- or combination therapy). No safety concerns were noted regarding laboratory values, electrocardiograms, and vital signs.

Detailed Results

Subject and Treatment Information

Study Completion/Withdrawal Information

TABLE 3
Subjects screened, randomized, and treated; all subjects.

| | Cmpd (1) | Cmpd (1) | Cmpd (1) | Cmpd (1) | | |
|---|---|---|---|---|---|---|
| | placebo | 300 mg | 600 mg | 600 mg | All | |
| | OST placebo | OST placebo | OST placebo | OST 75 mg | Subjects | |
| Screened | | | | | 967 | (100%) |
| Screen failure | | | | | 674 | (69.7%) |
| Randomized and/or | 72 | 74 | 74 | 72 | 293 | (30.3%) |
| treated | | | | | | |
| Not randomized | 0 | 1 | 0 | 0 | 675 | (69.8%) |
| Randomized, not | 0 | 0 | 0 | 0 | 1 | (0.1%) |
| treated | | | | | | |
| Treated, not | 0 | 1 | 0 | 0 | 1 | (0.1%) |
| randomized | | | | | | |
| Safety Set | 72 | 74 | 74 | 72 | 292 | (30.2%) |
| All subjects treated, | 20 | 16 | 17 | 16 | 69 | (7.1%) |
| without confirmed | | | | | | |
| influenza A | | | | | | |
| Full Analysis Set^{a} | 51 | 58 | 57 | 57 | 223 | (23.0%) |
| Intent-to-treat Set^{a} | 71 | 73 | 74 | 73 | 291 | (30.1%) |

^{a}For Full Analysis Set and Intent-to-treat Set, subjects were counted per treatment as randomized. For all other analysis sets subjects were counted per treatment as actually treated.

For one subject, treatment was initiated at the site without a proper randomization procedure; the subject was randomized after start of study medication. This subject was considered as treated but not randomized and was excluded from the Full Analysis Set (FAS), but included in the Safety Set.

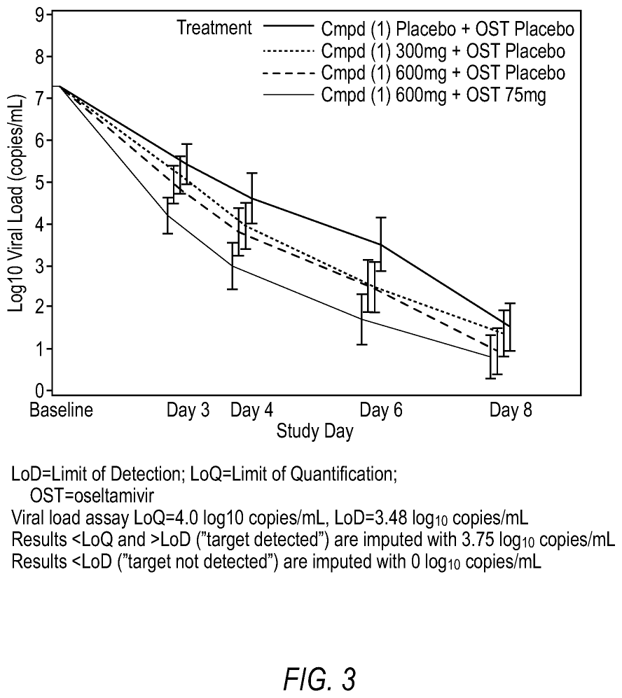

Referring to FIG. 3, the final analysis included data from 293 subjects. The majority of screening failures were due to a negative result for the rapid influenza diagnostic test. The safety set consisted of 292 subjects (one subject was not treated). The FAS consisted of 223 subjects that were randomized, treated, and confirmed influenza A positive.

Given the sizable difference between the FAS and the Safety Set, summary tables are provided for both analysis sets. Table 4 and Table 5 present a summary of subjects who completed or discontinued study drug and/or the study for the FAS and the Safety Set, respectively.

In the Compound (1) 300 mg twice daily (bid) treatment group, 20.7% of the subjects in the FAS discontinued treatment, compared to 7.8%, 14.0%, and 15.8% in the placebo, Compound (1) 600 mg bid, and Compound (1) 600 mg bid+oseltamivir 75 mg bid treatment groups, respectively.

TABLE 4
Completions and Discontinuations and Reasons for Discontinuation; Full Analysis Set.

| | Cmpd (1) | Cmpd (1) | | Cmpd (1) | | Cmpd (1) | | | |
|---|---|---|---|---|---|---|---|---|---|
| | placebo | 300 mg | | 600 mg | | 600 mg | | All | |
| | OST placebo | OST placebo | | OST placebo | | OST 75 mg | | Subjects | |
| Full Analysis Set | 51 | 58 | | 57 | | 57 | | 223 | |
| Drug termination | 51 | 58 | | 57 | | 57 | | 223 | |
| Completed | 47 (92.2%) | 46 | (79.3%) | 49 | (86.0%) | 48 | (84.2%) | 190 | (85.2%) |
| Discontinued | 4 (7.8%) | 12 | (20.7%) | 8 | (14.0%) | 9 | (15.8%) | 33 | (14.8%) |
| Adverse event | 3 (5.9%) | 6 | (10.3%) | 3 | (5.3%) | 5 | (8.8%) | 17 | (7.6%) |
| Physician decision | 1 (2.0%) | 3 | (5.2%) | 0 | | 1 | (1.8%) | 5 | (2.2%) |
| Withdrawal by subject | 0 | 2 | (3.4%) | 2 | (3.5%) | 1 | (1.8%) | 5 | (2.2%) |
| Other | 0 | 1 | (1.7%) | 3 | (5.3%) | 2 | (3.5%) | 6 | (2.7%) |
| Study termination | 51 | 58 | | 57 | | 57 | | 223 | |
| Completed | 49 (96.1%) | 54 | (93.1%) | 52 | (91.2%) | 55 | (96.5%) | 210 | (94.2%) |
| Discontinued | 2 (3.9%) | 4 | (6.9%) | 5 | (8.8%) | 2 | (3.5%) | 13 | (5.8%) |
| Protocol deviation | 0 | 1 | (1.7%) | 0 | | 0 | | 1 | (0.4%) |
| Withdrawal by subject | 1 (2.0%) | 2 | (3.4%) | 4 | (7.0%) | 1 | (1.8%) | 8 | (3.6%) |
| Other | 1 (2.0%) | 1 | (1.7%) | 1 | (1.8%) | 1 | (1.8%) | 4 | (1.8%) |

TABLE 5
Completions and discontinuations and reasons for discontinuation; safety set.

| | Cmpd (1) | Cmpd (1) | Cmpd (1) | Cmpd (1) | | |
|---|---|---|---|---|---|---|
| | placebo | 300 mg | 600 mg | 600 mg | All | |
| | OST placebo | OST placebo | OST placebo | OST 75 mg | Subjects | |
| Safety Set | 72 | 74 | 74 | 72 | 292 | |
| Drug termination | 72 | 74 | 74 | 72 | 292 | |
| Completed | 66 (91.7%) | 60 (81.1%) | 62 (83.8%) | 60 (83.3%) | 248 | (84.9%) |
| Discontinued | 6 (8.3%) | 14 (18.9%) | 12 (16.2%) | 12 (16.7%) | 44 | (15.1%) |
| Adverse event | 5 (6.9%) | 7 (9.5%) | 3 (4.1%) | 6 (8.3%) | 21 | (7.2%) |
| Non-compliance with | 0 | 0 | 0 | 1 (1.4%) | 1 | (0.3%) |
| study drug | | | | | | |
| Physician decision | 1 (1.4%) | 3 (4.1%) | 0 | 1 (1.4%) | 5 | (1.7%) |
| Withdrawal by subject | 0 | 3 (4.1%) | 4 (5.4%) | 1 (1.4%) | 8 | (2.7%) |
| Other | 0 | 1 (1.4%) | 5 (6.8%) | 3 (4.2%) | 9 | (3.1%) |
| Study termination | 72 | 74 | 74 | 72 | 292 | |
| Completed | 70 (97.2%) | 69 (93.2%) | 67 (90.5%) | 69 (95.8%) | 275 | (94.2%) |
| Discontinued | 2 (2.8%) | 5 (6.8%) | 7 (9.5%) | 3 (4.2%) | 17 | (5.8%) |
| Protocol deviation | 0 | 1 (1.4%) | 0 | 1 (1.4%) | 2 | (0.7%) |
| Withdrawal by subject | 1 (1.4%) | 3 (4.1%) | 6 (8.1%) | 1 (1.4%) | 11 | (3.8%) |
| Other | 1 (1.4%) | 1 (1.4%) | 1 (1.4%) | 1 (1.4%) | 4 | (1.4%) |

Demographic and Baseline Characteristics

Tables 6 and 7 present a summary of demographic characteristics for the FAS and the Safety Set, respectively. No notable differences between treatment groups were observed.

TABLE 6
Demographic characteristics; full analysis set.

| | Cmpd (1) | | Cmpd (1) | | Cmpd (1) | | Cmpd (1) | | | |
|---|---|---|---|---|---|---|---|---|---|---|
| | placebo | | 300 mg | | 600 mg | | 600 mg | | All | |
| | OST placebo | | OST placebo | | OST placebo | | OST 75 mg | | Subjects | |
| Full Analysis Set | 51 | | 58 | | 57 | | 57 | | 223 | |
| Age (Years) | | | | | | | | | | |
| N | 51 | | 58 | | 57 | | 57 | | 223 | |
| Mean (SD) | 40.1 | (12.23) | 41.3 | (13.32) | 37.1 | (14.11) | 42.0 | (12.92) | 40.1 | (13.23) |
| Median | 40.0 | | 42.0 | | 35.0 | | 43.0 | | 41.0 | |
| Range | (21; 63) | | (18; 64) | | (18; 65) | | (19; 64) | | (18; 65) | |
| BMI (kg/m²) | | | | | | | | | | |
| N | 51 | | 58 | | 57 | | 57 | | 223 | |
| Mean (SD) | 26.68 | (3.948) | 26.09 | (4.230) | 26.76 | (4.594) | 27.08 | (3.714) | 26.65 | (4.128) |
| Median | 25.20 | | 25.35 | | 26.90 | | 27.00 | | 26.60 | |
| Range | (20.0; 35.0) | | (18.6; 34.8) | | (19.0; 35.0) | | (19.0; 33.5) | | (18.6; 35.0) | |
| Country | | | | | | | | | | |
| N | 51 | | 58 | | 57 | | 57 | | 223 | |
| Belgium | 3 | (5.9%) | 6 | (10.3%) | 4 | (7.0%) | 3 | (5.3%) | 16 | (7.2%) |
| Bulgaria | 0 | | 0 | | 2 | (3.5%) | 1 | (1.8%) | 3 | (1.3%) |
| Canada | 1 | (2.0%) | 2 | (3.4%) | 2 | (3.5%) | 2 | (3.5%) | 7 | (3.1%) |
| Estonia | 4 | (7.8%) | 7 | (12.1%) | 5 | (8.8%) | 5 | (8.8%) | 21 | (9.4%) |
| Latvia | 1 | (2.0%) | 1 | (1.7%) | 0 | | 1 | (1.8%) | 3 | (1.3%) |
| South Africa | 0 | | 0 | | 1 | (1.8%) | 0 | | 1 | (0.4%) |
| United States | 42 | (82.4%) | 42 | (72.4%) | 43 | (75.4%) | 45 | (78.9%) | 172 | (77.1%) |
| Gender | | | | | | | | | | |
| N | 51 | | 58 | | 57 | | 57 | | 223 | |
| Female | 24 | (47.1%) | 28 | (48.3%) | 30 | (52.6%) | 31 | (54.4%) | 113 | (50.7%) |
| Male | 27 | (52.9%) | 30 | (51.7%) | 27 | (47.4%) | 26 | (45.6%) | 110 | (49.3%) |
| Race | | | | | | | | | | |
| N | 51 | | 58 | | 57 | | 57 | | 223 | |
| White | 46 | (90.2%) | 44 | (75.9%) | 48 | (84.2%) | 50 | (87.7%) | 188 | (84.3%) |
| Black or | 4 | (7.8%) | 8 | (13.8%) | 6 | (10.5%) | 3 | (5.3%) | 21 | (9.4%) |
| African American | | | | | | | | | | |
| Asian | 1 | (2.0%) | 6 | (10.3%) | 3 | (5.3%) | 4 | (7.0%) | 14 | (6.3%) |
| Ethnicity, n (%) | | | | | | | | | | |
| N | 51 | | 58 | | 57 | | 57 | | 223 | |
| Hispanic | 10 | (19.6%) | 11 | (19.0%) | 12 | (21.1%) | 12 | (21.1%) | 45 | (20.2%) |
| or Latino | | | | | | | | | | |
| Not Hispanic | 41 | (80.4%) | 47 | (81.0%) | 45 | (78.9%) | 45 | (78.9%) | 178 | (79.8%) |
| or Latino | | | | | | | | | | |

N = number of subjects with data;
SD = standard deviation

TABLE 7
Demographic characteristics; safety set.

| | Cmpd (1) | | Cmpd (1) | | Cmpd (1) | | Cmpd (1) | | | |
|---|---|---|---|---|---|---|---|---|---|---|
| | placebo + | | 300 mg + | | 600 mg + | | 600 mg + | | | |
| | OST placebo | | OST placebo | | OST placebo | | OST 75 mg | | Total | |
| Analysis Set: | 72 | | 74 | | 74 | | 72 | | 292 | |
| Safety Set | | | | | | | | | | |
| Age (Years) | | | | | | | | | | |
| N | 72 | | 74 | | 74 | | 72 | | 292 | |
| Mean (SD) | 41.7 | (12.42) | 41.6 | (12.79) | 37.6 | (13.49) | 40.8 | (13.00) | 40.4 | (12.98) |
| Median | 43.5 | | 43.0 | | 37.0 | | 43.0 | | 42.0 | |
| Range | (18; 64) | | (18; 64) | | (18; 65) | | (19; 64) | | (18; 65) | |
| BMI (kg/m²) | | | | | | | | | | |
| N | 72 | | 74 | | 74 | | 72 | | 292 | |
| Mean (SD) | 26.55 | (3.886) | 26.33 | (4.124) | 26.77 | (4.414) | 27.10 | (3.888) | 26.69 | (4.075) |
| Median | 25.85 | | 26.10 | | 26.90 | | 27.05 | | 26.60 | |
| Range | (19.6; 35.0) | | (18.6; 34.8) | | (19.0; 35.0) | | (18.1; 33.5) | | (18.1; 35.0) | |
| Country | | | | | | | | | | |
| N | 72 | | 74 | | 74 | | 72 | | 292 | |
| Belgium | 4 | (5.6%) | 7 | (9.5%) | 4 | (5.4%) | 4 | (5.6%) | 19 | (6.5%) |
| Bulgaria | 0 | | 0 | | 2 | (2.7%) | 1 | (1.4%) | 3 | (1.0%) |
| Canada | 1 | (1.4%) | 2 | (2.7%) | 2 | (2.7%) | 2 | (2.8%) | 7 | (2.4%) |
| Estonia | 4 | (5.6%) | 7 | (9.5%) | 5 | (6.8%) | 5 | (6.9%) | 21 | (7.2%) |
| Latvia | 2 | (2.8%) | 1 | (1.4%) | 0 | | 1 | (1.4%) | 4 | (1.4%) |
| South Africa | 3 | (4.2%) | 2 | (2.7%) | 2 | (2.7%) | 1 | (1.4%) | 8 | (2.7%) |
| United States | 58 | (80.6%) | 55 | (74.3%) | 59 | (79.7%) | 58 | (80.6%) | 230 | (78.8%) |
| Gender | | | | | | | | | | |
| N | 72 | | 74 | | 74 | | 72 | | 292 | |
| Female | 32 | (44.4%) | 35 | (47.3%) | 41 | (55.4%) | 42 | (58.3%) | 150 | (51.4%) |
| Male | 40 | (55.6%) | 39 | (52.7%) | 33 | (44.6%) | 30 | (41.7%) | 142 | (48.6%) |
| Race | | | | | | | | | | |
| N | 72 | | 74 | | 74 | | 72 | | 292 | |
| White | 61 | (84.7%) | 57 | (77.0%) | 64 | (86.5%) | 63 | (87.5%) | 245 | (83.9%) |
| Black or | 9 | (12.5%) | 10 | (13.5%) | 6 | (8.1%) | 5 | (6.9%) | 30 | (10.3%) |
| African American | | | | | | | | | | |
| Asian | 2 | (2.8%) | 6 | (8.1%) | 4 | (5.4%) | 4 | (5.6%) | 16 | (5.5%) |
| Other | 0 | | 1 | (1.4%) | 0 | | 0 | | 1 | (0.3%) |
| Ethnicity | | | | | | | | | | |
| N | 72 | | 74 | | 74 | | 72 | | 292 | |
| Hispanic | 19 | (26.4%) | 22 | (29.7%) | 26 | (35.1%) | 25 | (34.7%) | 92 | (31.5%) |
| or Latino | | | | | | | | | | |
| Not Hispanic | 53 | (73.6%) | 52 | (70.3%) | 48 | (64.9%) | 47 | (65.3%) | 200 | (68.5%) |
| or Latino | | | | | | | | | | |

BMI = body mass index;
N = number of subjects with data;
SD = standard deviation

Tables 8 and 9 present a summary of baseline disease characteristics for the FAS and the Safety Set, respectively.

TABLE 8
Baseline disease characteristics; full analysis set.

| | Cmpd (1) | | Cmpd (1) | | Cmpd (1) | | Cmpd (1) | | | |
|---|---|---|---|---|---|---|---|---|---|---|
| | placebo + | | 300 mg + | | 600 mg + | | 600 mg + | | | |
| | OST placebo | | OST placebo | | OST placebo | | OST 75 mg | | Total | |
| Analysis Set: | 51 | | 58 | | 57 | | 57 | | 223 | |
| Full Analysis Set | | | | | | | | | | |
| Average of the 7 | | | | | | | | | | |
| primary influenza | | | | | | | | | | |
| symptom scores | | | | | | | | | | |
| N | 51 | | 58 | | 57 | | 57 | | 223 | |
| Mean (SD) | 2.07 | (0.401) | 2.11 | (0.449) | 2.19 | (0.401) | 2.14 | (0.462) | 2.13 | (0.429) |
| Median | 2.14 | | 2.14 | | 2.14 | | 2.29 | | 2.14 | |
| Range | (1.3; 2.9) | | (1.3; 3.0) | | (1.0; 3.0) | | (1.0; 3.0) | | (1.0; 3.0) | |
| Temperature (° C.) | | | | | | | | | | |
| N | 51 | | 58 | | 57 | | 57 | | 223 | |
| Mean (SD) | 38.00 | (0.827) | 38.13 | (0.628) | 37.94 | (0.778) | 38.04 | (0.679) | 38.03 | (0.727) |
| Median | 38.10 | | 38.20 | | 38.10 | | 38.20 | | 38.20 | |
| Range | (35.2; 39.8) | | (36.7; 39.0) | | (36.2; 39.4) | | (36.2; 39.4) | | (35.2; 39.8) | |
| Influenza A viral | | | | | | | | | | |
| load by qRT-PCR | | | | | | | | | | |
| (log₁₀ (copies/mL)) | | | | | | | | | | |
| N | 51 | | 57 | | 56 | | 57 | | 221 | |
| Mean (SD) | 6.84 | (2.068) | 7.58 | (1.207) | 7.48 | (1.084) | 7.16 | (1.326) | 7.28 | (1.472) |
| Median | 7.32 | | 7.65 | | 7.62 | | 7.55 | | 7.54 | |
| Range | (0.0; 9.5) | | (4.6; 9.9) | | (4.2; 9.4) | | (4.1; 9.5) | | (0.0; 9.9) | |
| Influenza A viral | | | | | | | | | | |
| load by viral culture | | | | | | | | | | |
| (log₁₀ (TCID₅₀/mL)) | | | | | | | | | | |
| N | 51 | | 57 | | 56 | | 56 | | 220 | |
| Mean (SD) | 3.18 | (1.850) | 3.40 | (1.658) | 3.39 | (1.689) | 3.04 | (1.653) | 3.26 | (1.706) |
| Median | 3.50 | | 3.50 | | 3.38 | | 3.25 | | 3.38 | |
| Range | (0.4; 6.0) | | (0.4; 6.3) | | (0.4; 6.3) | | (0.4; 6.5) | | (0.4; 6.5) | |
| Intensity of Fever | | | | | | | | | | |
| N | 51 | | 58 | | 57 | | 57 | | 223 | |
| Mild | 10 | (19.6%) | 8 | (13.8%) | 9 | (15.8%) | 10 | (17.5%) | 37 | (16.6%) |
| Moderate | 26 | (51.0%) | 25 | (43.1%) | 31 | (54.4%) | 28 | (49.1%) | 110 | (49.3%) |
| Severe | 15 | (29.4%) | 25 | (43.1%) | 17 | (29.8%) | 19 | (33.3%) | 76 | (34.1%) |
| Onset of Fever | | | | | | | | | | |
| N | 51 | | 58 | | 57 | | 57 | | 223 | |
| ≤24 hours | 22 | (43.1%) | 27 | (46.6%) | 30 | (52.6%) | 28 | (49.1%) | 107 | (48.0%) |
| Within 24-≤48 | 29 | (56.9%) | 31 | (53.4%) | 27 | (47.4%) | 29 | (50.9%) | 116 | (52.0%) |
| hours | | | | | | | | | | |
| Time Since Onset of | | | | | | | | | | |
| Symptoms | | | | | | | | | | |
| N | 51 | | 58 | | 57 | | 57 | | 223 | |
| ≤24 hours | 22 | (43.1%) | 24 | (41.4%) | 23 | (40.4%) | 25 | (43.9%) | 94 | (42.2%) |
| Within 24-≤48 | 29 | (56.9%) | 34 | (58.6%) | 34 | (59.6%) | 32 | (56.1%) | 129 | (57.8%) |
| hours | | | | | | | | | | |

N = number of subjects with data;
qRT-PCR = quantitative reverse transcriptase polymerase chain reaction;
SD = standard deviation;
TCID₅₀ = median tissue culture infective dose

TABLE 9
Baseline disease characteristics; safety set.

| | Cmpd (1) | | Cmpd (1) | | Cmpd (1) | | Cmpd (1) | | | |
|---|---|---|---|---|---|---|---|---|---|---|
| | placebo + | | 300 mg + | | 600 mg + | | 600 mg + | | | |
| | OST placebo | | OST placebo | | OST placebo | | OST 75 mg | | Total | |
| Analysis Set: | 72 | | 74 | | 74 | | 72 | | 292 | |
| Safety Analysis Set | | | | | | | | | | |
| Total of the 7 | | | | | | | | | | |
| primary influenza | | | | | | | | | | |
| symptom scores | | | | | | | | | | |
| N | 72 | | 74 | | 74 | | 72 | | 292 | |
| Mean (SD) | 2.05 | (0.449) | 2.12 | (0.480) | 2.23 | (0.453) | 2.21 | (0.448) | 2.15 | (0.461) |
| Median | 2.00 | | 2.14 | | 2.21 | | 2.29 | | 2.14 | |
| Range | (1.3; 3.0) | | (0.7; 3.0) | | (0.9; 3.0) | | (1.0; 3.0) | | (0.7; 3.0) | |
| Temperature (° C.) | | | | | | | | | | |
| N | 72 | | 74 | | 74 | | 72 | | 292 | |
| Mean (SD) | 37.93 | (0.835) | 38.09 | (0.635) | 37.96 | (0.758) | 38.08 | (0.712) | 38.01 | (0.738) |
| Median | 38.10 | | 38.20 | | 38.10 | | 38.20 | | 38.10 | |
| Range | (35.2; 39.8) | | (36.6; 39.2) | | (36.2; 39.4) | | (36.2; 40.3) | | (35.2; 40.3) | |
| Intensity of Fever | | | | | | | | | | |
| N | 72 | | 74 | | 74 | | 72 | | 292 | |
| Mild | 12 | (16.7%) | 9 | (12.2%) | 11 | (14.9%) | 12 | (16.7%) | 44 | (15.1%) |
| Moderate | 38 | (52.8%) | 30 | (40.5%) | 41 | (55.4%) | 34 | (47.2%) | 143 | (49.0%) |
| Severe | 22 | (30.6%) | 35 | (47.3%) | 22 | (29.7%) | 26 | (36.1%) | 105 | (36.0%) |
| Onset of Fever | | | | | | | | | | |
| N | 72 | | 74 | | 74 | | 72 | | 292 | |
| ≤24 hours | 31 | (43.1%) | 35 | (47.3%) | 38 | (51.4%) | 34 | (47.2%) | 138 | (47.3%) |
| Within 24-≤48 | 41 | (56.9%) | 39 | (52.7%) | 36 | (48.6%) | 38 | (52.8%) | 154 | (52.7%) |
| hours | | | | | | | | | | |
| Time Since Onset of | | | | | | | | | | |
| Symptoms | | | | | | | | | | |
| N | 72 | | 74 | | 74 | | 72 | | 292 | |
| ≤24 hours | 30 | (41.7%) | 32 | (43.2%) | 32 | (43.2%) | 32 | (44.4%) | 126 | (43.2%) |
| Within 24-≤48 | 42 | (58.3%) | 42 | (56.8%) | 42 | (56.8%) | 40 | (55.6%) | 166 | (56.8%) |
| hours | | | | | | | | | | |

N = number of subjects with data;
SD = standard deviation

Antipyretic Concomitant Medications

Table 10 presents a summary of antipyretic concomitant medication use for the FAS.

TABLE 10
Antipyretic concomitant medications; full analysis set.

| | Cmpd (1) | Cmpd (1) | Cmpd (1) | Cmpd (1) | | |
|---|---|---|---|---|---|---|
| | placebo + | 300 mg + | 600 mg + | 600 mg + | | |
| | OST placebo | OST placebo | OST placebo | OST 75 mg | Total | |
| Full Analysis Set | 51 | 58 | 57 | 57 | 223 | |
| Any antipyretic | 44 (86.3%) | 44 (75.9%) | 46 (80.7%) | 47 (82.5%) | 181 | (81.2%) |
| concomitant medication | | | | | | |
| Ibuprofen | 20 (39.2%) | 19 (32.8%) | 23 (40.4%) | 20 (35.1%) | 82 | (36.8%) |
| Paracetamol | 33 (64.7%) | 38 (65.5%) | 30 (52.6%) | 37 (64.9%) | 138 | (61.9%) |
| Other | 11 (21.6%) | 17 (29.3%) | 12 (21.1%) | 12 (21.1%) | 52 | (23.3%) |

Medication received at or after the first dose of study drug, medication that was received before initial dosing and continued after initial dosing of study drug, or medication with missing stop date.

Antibiotics Related to Influenza Complications

Table 11 presents a summary of antibiotics related to influenza complications for the FAS.

TABLE 11
Antibiotics related to influenza complications; full analysis set.

| | Cmpd (1) | Cmpd (1) | Cmpd (1) | Cmpd (1) | |
|---|---|---|---|---|---|
| | placebo + | 300 mg + | 600 mg + | 600 mg + | |
| | OST placebo | OST placebo | OST placebo | OST 75 mg | Total |
| Full Analysis Set | 51 | 58 | 57 | 57 | 223 |
| Any antibiotic related | 2 (3.9%) | 2 (3.4%) | 0 | 0 | 4 (1.8%) |
| to influenza complications | | | | | |
| Amoxi-Clavulanico | 1 (2.0%) | 0 | 0 | 0 | 1 (0.4%) |
| Azithromycin | 0 | 1 (1.7%) | 0 | 0 | 1 (0.4%) |
| Bactrim | 1 (2.0%) | 1 (1.7%) | 0 | 0 | 2 (0.9%) |

Extent of Exposure

Table 12 and Table 13 present a summary of the extent of exposure and compliance for the FAS and the Safety Set, respectively. Compliance is expressed on the scale from 0 to 100, with 100 being fully compliant, i.e. having taken all medication. The compliance to tablet and capsule intake was markedly higher in the placebo group versus the active groups.

TABLE 12
Extent of exposure and compliance; full analysis set.

| | Cmpd (1) | | Cmpd (1) | | Cmpd (1) | | Cmpd (1) | | | |
|---|---|---|---|---|---|---|---|---|---|---|
| | placebo + | | 300 mg + | | 600 mg + | | 600 mg + | | | |
| | OST placebo | | OST placebo | | OST placebo | | OST 75 mg | | Total | |
| Analysis Set: | 51 | | 58 | | 57 | | 57 | | 223 | |
| Full Analysis Set | | | | | | | | | | |
| Extent of | | | | | | | | | | |
| exposure (hours) | | | | | | | | | | |
| N | 51 | | 58 | | 57 | | 57 | | 223 | |
| Mean (SD) | 116.3 | (16.38) | 110.9 | (26.83) | 114.9 | (26.03) | 113.9 | (26.38) | 113.9 | (24.41) |
| Median | 119.7 | | 119.7 | | 120.8 | | 119.4 | | 119.6 | |
| Range | (44; 145) | | (12; 142) | | (12; 144) | | (31; 171) | | (12; 171) | |
| Compliance Cmpd | | | | | | | | | | |
| (1)/Placebo | | | | | | | | | | |
| N | 50 | | 58 | | 57 | | 57 | | 222 | |
| Mean (SD) | 96.70 | (13.310) | 89.31 | (23.159) | 93.33 | (20.052) | 92.11 | (20.937) | 92.73 | (19.935) |
| Median | 100.00 | | 100.00 | | 100.00 | | 100.00 | | 100.00 | |
| Range | (40.0; 100.0) | | (10.0; 100.0) | | (10.0; 100.0) | | (20.0; 100.0) | | (10.0; 100.0) | |
| Compliance | | | | | | | | | | |
| Oseltamivir/ | | | | | | | | | | |
| Placebo | | | | | | | | | | |
| N | 50 | | 58 | | 57 | | 57 | | 222 | |
| Mean (SD) | 97.00 | (12.495) | 89.31 | (23.159) | 93.33 | (20.119) | 92.28 | (20.356) | 92.84 | (19.694) |

| | | | | | |
|---|---|---|---|---|---|
| Median | 100.00 | 100.00 | 100.00 | 100.00 | 100.00 |
| Range | (40.0; 100.0) | (10.0; 100.0) | (10.0; 100.0) | (30.0; 100.0) | (10.0; 100.0) |

N = number of subjects with data
SD = standard deviation
Extent of exposure: treatment duration is defined as datetime of last study drug intake − datetime of first study drug intake + 12 hours.

TABLE 13
Extent of exposure and compliance; safety set.

| | Cmpd (1) | | Cmpd (1) | | Cmpd (1) | | Cmpd (1) | | | |
|---|---|---|---|---|---|---|---|---|---|---|
| | placebo + | | 300 mg + | | 600 mg + | | 600 mg + | | | |
| | OST placebo | | OST placebo | | OST placebo | | OST 75 mg | | Total | |
| Analysis Set: | 72 | | 74 | | 74 | | 72 | | 292 | |
| Safety Set | | | | | | | | | | |
| Extent of | | | | | | | | | | |
| exposure (hours) | | | | | | | | | | |
| N | 72 | | 74 | | 74 | | 72 | | 292 | |
| Mean (SD) | 118.6 | (16.77) | 112.7 | (25.43) | 112.6 | (28.96) | 113.0 | (26.01) | 114.2 | (24.75) |
| Median | 120.0 | | 120.0 | | 120.6 | | 119.5 | | 119.9 | |
| Range | (44; 188) | | (12; 144) | | (12; 144) | | (31; 171) | | (12; 188) | |
| Compliance Cmpd | | | | | | | | | | |
| (1)/Placebo | | | | | | | | | | |
| N | 71 | | 74 | | 74 | | 72 | | 291 | |
| Mean (SD) | 96.69 | (12.732) | 90.14 | (22.237) | 91.35 | (22.365) | 91.25 | (21.750) | 92.32 | (20.292) |
| Median | 100.00 | | 100.00 | | 100.00 | | 100.00 | | 100.00 | |
| Range | (40.0; 100.0) | | (10.0; 100.0) | | (10.0; 100.0) | | (20.0; 100.0) | | (10.0; 100.0) | |
| Compliance | | | | | | | | | | |
| Oseltamivir/ | | | | | | | | | | |
| Placebo | | | | | | | | | | |
| N | 71 | | 74 | | 74 | | 72 | | 291 | |
| Mean (SD) | 96.90 | (12.141) | 90.14 | (22.237) | 91.35 | (22.411) | 91.39 | (21.316) | 92.41 | (20.113) |

| | | | | | |
|---|---|---|---|---|---|
| Median | 100.00 | 100.00 | 100.00 | 100.00 | 100.00 |
| Range | (40.0; 100.0) | (10.0; 100.0) | (10.0; 100.0) | (30.0; 100.0) | (10.0; 100.0) |

N = number of subjects with data;
SD = standard deviation
Extent of exposure: treatment duration is defined as datetime of last study drug intake − datetime of first study drug intake + 12 hours.

Primary Endpoint Analysis

The primary endpoint was viral load area under the curve (AUC) from Day 1 to Day 8 as measured by quantitative reverse transcriptase polymerase chain reaction (qRT-PCR).

The primary analysis results are presented in Table 12 and show a statistically significant dose-response relationship: p values of 0.009 and 0.010 (adjusted for multiplicity) for monotherapies combined versus placebo and a linear dose-response trend, respectively, as compared to the 1-sided type I error limit of 0.016. Adjustments were made for having multiple comparisons and performing an interim look to evaluate the primary study objective.

The average reduction on the AUC of viral load versus placebo treatment was estimated to be 3.6 (p=0.044), −4.5 (p=0.012), and −8.6 (p<0.001) day*log10 copies/mL for the Compound (1) 300 mg bid, Compound (1) 600 mg bid and Compound (1) 600 mg bid+oseltamivir 75 mg bid treatment groups, respectively (Table 12). The average reduction in AUC viral load of Compound (1) 600 mg bid+oseltamivir 75 mg bid versus Compound (1) 600 mg bid treatment was 4.1 (p=0.017).

The estimated least square (LS) Means and 95% confidence intervals (CIs) for viral load measured by qRT-PCR at each visit, and the estimated differences in AUC viral load measured by qRT-PCR between the active treatment groups and placebo are shown in Table 14. FIG. 3 is a graphical representation of the estimated LS Means and 95% CIs for viral load over time.

TABLE 14
Primary Endpoint - Viral Shedding: Viral Area Under the Curve (AUC):
Linear Model; Full Analysis Set

| | LS Means (95% CI) | | | | Estimate | p |
|---|---|---|---|---|---|---|
| Treatment | Day 3 | Day 4 | Day 6 | Day 8 | (95% CI) | value |
| | | | | | Difference vs | |
| | | | | | Placebo in Viral | |
| | | | | | Load AUC Day 1 to | |
| | | | | | 8 (day * log₁₀ | |
| | | | | | copies/mL) | |
| Cmpd (1) placebo + | 5.4 | 4.6 | 3.5 | 1.5 | | |
| OST placebo | (4.9; 5.9) | (4.0; 5.2) | (2.9; 4.1) | (1.0; 2.1) | | |
| Cmpd (1) 300 mg + | 5.2 | 3.9 | 2.5 | 1.4 | −3.6 | 0.044 |
| OST placebo | (4.7; 5.6) | (3.4; 4.5) | (1.9; 3.1) | (0.8; 1.9) | (−7.1; −0.1) | |
| Cmpd (1) 600 mg + | 4.9 | 3.8 | 2.5 | 1.0 | −4.5 | 0.012 |
| OST placebo | (4.5; 5.4) | (3.2; 4.4) | (1.9; 3.1) | (0.4; 1.5) | (−8.0; −1.0) | |
| Cmpd (1) 600 mg + | 4.2 | 3.0 | 1.7 | 0.8 | −8.6 | <.001 |
| OST 75 mg | (3.8; 4.6) | (2.4; 3.5) | (1.1; 2.3) | (0.3; 1.3) | (−12.0; −5.1) | |
| Dose-response | | | | | | |
| testing | | | | | | |
| Mono-therapies | | | | | −4.0 | 0.008* |
| combined | | | | | (−∞; −0.7) | |
| Linear trend | | | | | −4.5 | 0.010* |
| | | | | | (−∞; −0.6) | |

| | Difference vs Cmpd | |
|---|---|---|
| | (1) 600 mg in Viral | |
| | Load AUC Day 1 to | |
| | 8 (day * log₁₀ | |
| | copies/mL) | |
| Cmpd (1) 600 mg + | −4.1 | 0.017 |
| OST 75 mg | (−7.4; −0.75) | |

AUC = area under the curve
*The significance level for the difference between active and placebo is 0.016. A Pocock-type alpha spending function was used to control the overall family-wise Type 1 error rate at the 5% level taking into account 1 interim analysis and one final analysis after enrolment of total planned number of patients (2 analyses in total). The Pocock function is .025 * ln (1 + (e − 1) * t)) where t = n/N, n is the FA set size and N is the expected FA set size at the planned end of study (=428).

Key Secondary Endpoint Analysis

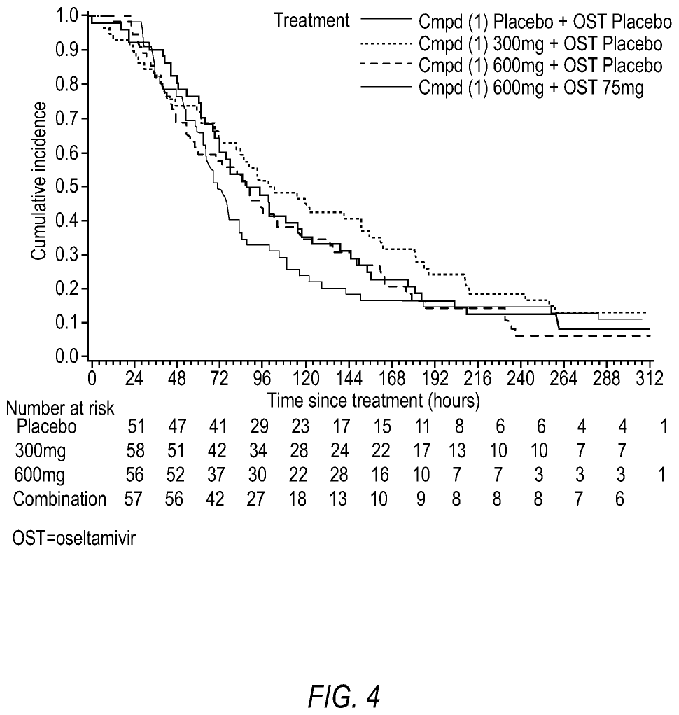

Kaplan-Meier curves of time to resolution of influenza symptoms by treatment group are provided in FIG. 4.

Estimated hazard ratios and results for the 2-sided log-rank and the Gehan-Wilcoxon tests comparing each active treatment versus placebo are shown in Table 15.

TABLE 15
Log rank test and hazard ratios for the time to resolution
of 7 primary influenza symptoms; full analysis set.

| | Hazard ratio | | | Gehan- |
|---|---|---|---|---|
| | | 95% | Log Rank | Wilcoxon |
| Treatment versus | | Confidence | Test | Test |
| Placebo | Estimate | Interval | p-value | p-value |
| Cmpd (1) 300 mg + | 0.84 | (0.56; 1.27) | 0.396 | 0.686 |
| OST Placebo | | | | |
| Cmpd (1) 600 mg + | 1.17 | (0.77; 1.75) | 0.583 | 0.468 |
| OST Placebo | | | | |
| Cmpd (1) 600 mg + | 1.20 | (0.80; 1.81) | 0.491 | 0.259 |
| OST 75 mg | | | | |

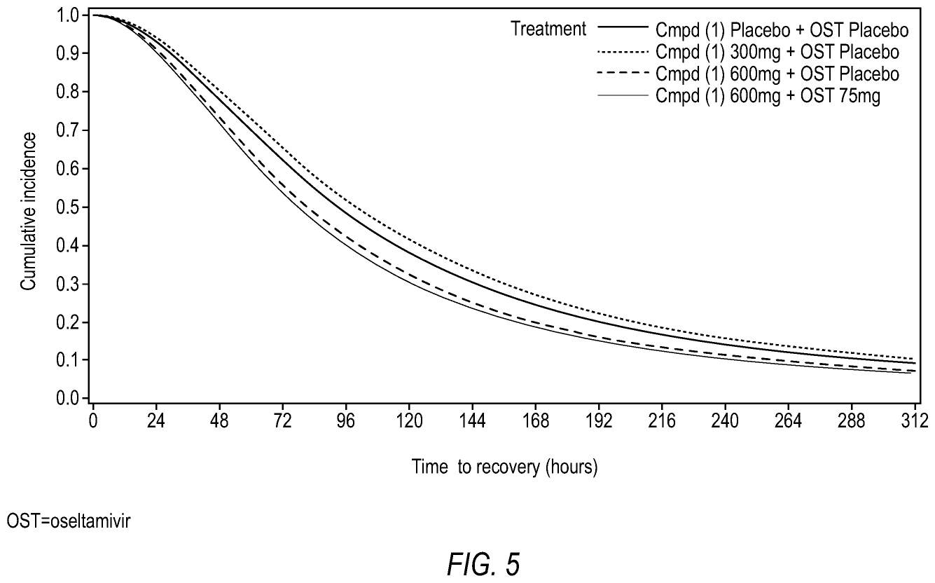

The estimated survival curves of the time to resolution of the 7 primary influenza symptoms, using an accelerated failure time model, are shown in FIG. 5 based on the mean baseline influenza symptom score and weighted average for stratum.

Estimated acceleration factors (expressed as the ratio of the time to resolution as compared to placebo) show that there was an increase of time to resolution of influenza symptoms versus placebo treatment of 7% for the Compound (1) 300 mg bid treatment group and a reduction of time to resolution of influenza symptoms versus placebo treatment of 13% and 17% for the Compound (1) 600 mg bid and Compound (1) 600 mg bid+oseltamivir 75 mg bid treatment groups, respectively (Table).

TABLE 16
Key secondary endpoint: Accelerated failure time model of the time to
resolution of the 7 primary influenza symptoms; full analysis set.

| | Acceleration | 95% Confidence |
|---|---|---|
| Treatment versus Placebo | Factor | Interval |
| Cmpd (1) 300 mg + OST Placebo | 1.07 | (0.76; 1.52) |
| Cmpd (1) 600 mg + OST Placebo | 0.87 | (0.62; 1.23) |
| Cmpd (1) 600 mg + OST 75 mg | 0.83 | (0.60; 1.16) |

Secondary Endpoint Analyses

Categorized Viral Load Data Over Time (qRT-PCR)

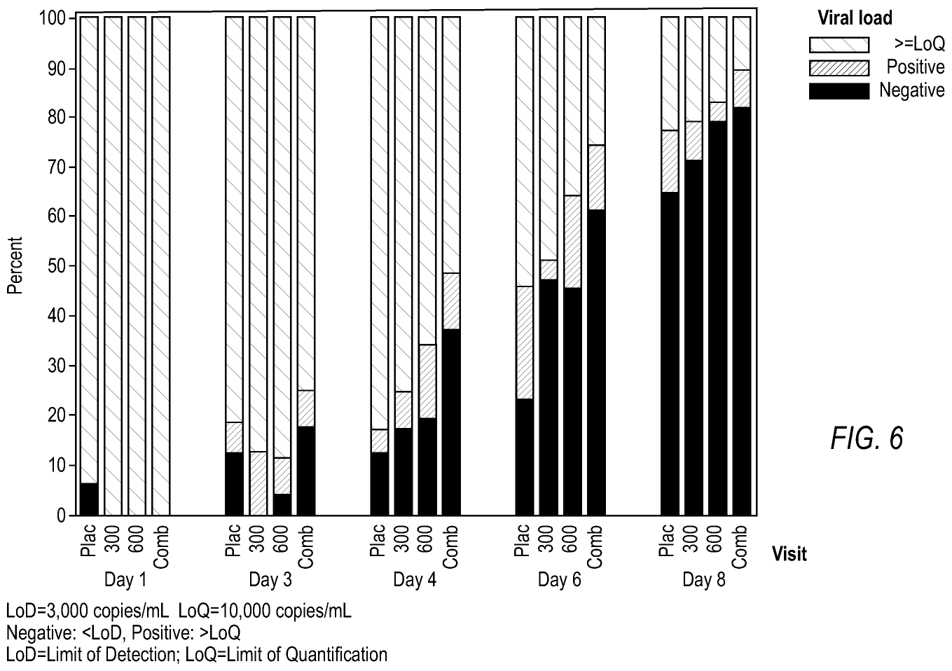

Percentages of subjects with viral load (qRT-PCR) categorized as negative (target not detected), positive (target detected), and ≥limit of quantification are presented in FIG. 6 for each visit and treatment group.

Duration of Viral Shedding (qRT-PCR)

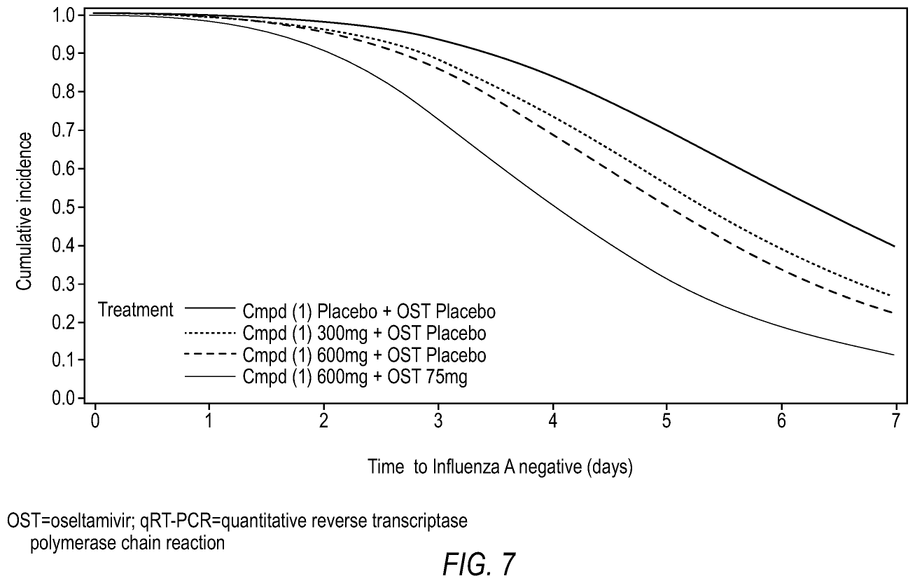

The estimated survival curves for time to negativity of qRT-PCR are shown in FIG. 7 based on the mean baseline viral load and weighted average for stratum.

Estimated acceleration factors (expressed as the ratio of the time to negativity as compared to placebo time to negativity) show there was a reduction in time to negative viral load versus placebo treatment of 13%, 18%, and 31% for the Compound (1) 300 mg bid, Compound (1) 600 mg bid, and Compound (1) 600 mg bid+oseltamivir 75 mg bid treatment groups, respectively (Table 17).

TABLE 17
Time to influenza A negativity (qRT-PCR): Accelerated
failure time model; full analysis set.

| | Acceleration | 95% Confidence |
|---|---|---|
| Treatment versus Placebo | Factor | Interval |
| Cmpd (1) 300 mg + OST Placebo | 0.87 | (0.74; 1.02) |
| Cmpd (1) 600 mg + OST Placebo | 0.82 | (0.70; 0.97) |
| Cmpd (1) 600 mg + OST 75 mg | 0.69 | (0.58; 0.81) |

qRT-PCR = quantitative reverse transcriptase polymerase chain reaction

Categorized Viral Load Data Over Time (Viral Culture)

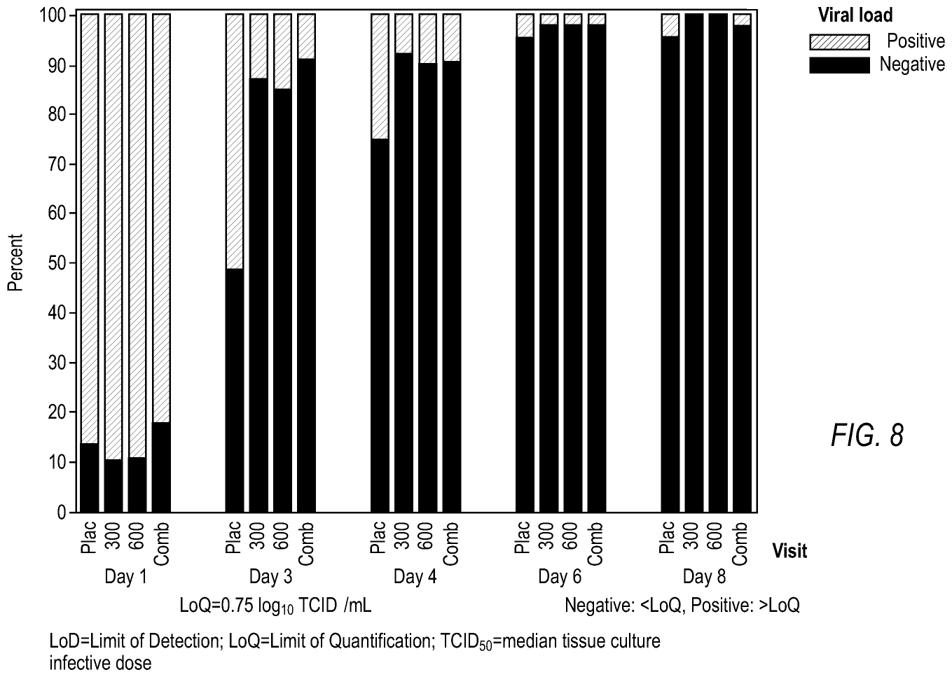

Percentages of subjects with viral load (viral culture) categorized as negative and positive are presented in FIG. 8 for each visit and treatment group.

Duration of Viral Shedding by Viral Culture

Estimated acceleration factors (expressed as the ratio of the time to negativity as compared to placebo time to negativity) show there was a reduction in time to negative viral load versus placebo treatment of 29%, 28%, and 37% for the Compound (1) 300 mg bid, Compound (1) 600 mg bid, and Compound (1) 600 mg bid+oseltamivir 75 mg bid treatment groups, respectively (see Table 18).

TABLE 18
Time to Influenza A Negativity (TCID₅₀): Accelerated
Failure Time Model; Full Analysis Set.

| | Acceleration | 95% Confidence |
|---|---|---|
| Treatment versus Placebo | Factor | Interval |
| Cmpd (1) 300 mg + OST Placebo | 0.71 | (0.58; 0.86) |
| Cmpd (1) 600 mg + OST Placebo | 0.72 | (0.60; 0.87) |
| Cmpd (1) 600 mg + OST 75 mg | 0.63 | (0.50; 0.78) |

TCID₅₀ = median tissue culture infective dose

Time to Resolution of Fever

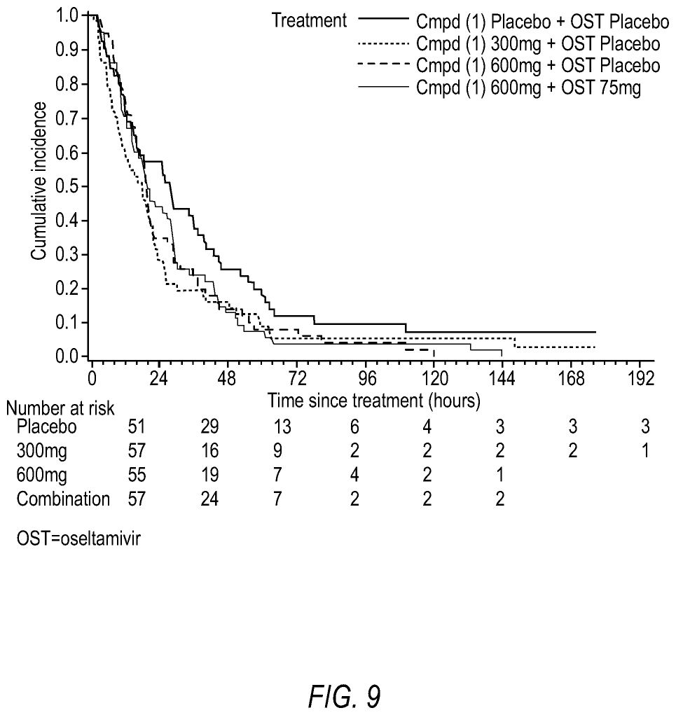

Time to resolution of fever defined as the time in hours from the first dose of investigational product until the time temperature equals or becomes lower than 37.2° C. (99.0° F.). Kaplan-Meier curves of time to resolution of fever by treatment group are provided in FIG. 9.

Safety

Summary of All Adverse Events

TABLE 19
Treatment-emergent Adverse Events Summary Table; Safety Set.

| | Cmpd (1) | Cmpd (1) | | Cmpd (1) | Cmpd (1) |
|---|---|---|---|---|---|
| | placebo | 300 mg | | 600 mg | 600 mg |
| | OST placebo | OST placebo | | OST placebo | OST 75 mg |
| Safety Set | 72 | 74 | | 74 | 72 |
| Any TEAE | 32 (44.4%) | 32 | (43.2%) | 39 (52.7%) | 29 (40.3%) |
| Any Serious TEAE | 1 (1.4%) | 0 | | 1 (1.4%) | 0 |
| Any Serious TEAE at least possibly | 1 (1.4%) | 0 | | 0 | 0 |
| related to study medication | | | | | |
| Any TEAE with fatal outcome | 0 | 0 | | 0 | 0 |
| Worst grade 1 or 2 TEAE | 28 (38.9%) | 30 | (40.5%) | 34 (45.9%) | 26 (36.1%) |
| Worst grade 3 or 4 TEAE | 4 (5.6%) | 2 | (2.7%) | 5 (6.8%) | 3 (4.2%) |
| Worst grade 3 TEAE | 4 (5.6%) | 2 | (2.7%) | 5 (6.8%) | 3 (4.2%) |
| Worst grade 4 TEAE | 0 | 0 | | 0 | 0 |
| Any TEAE at least possibly related | 13 (18.1%) | 9 | (12.2%) | 26 (35.1%) | 17 (23.6%) |
| to study medication | | | | | |
| Any TEAE leading to permanent | 5 (6.9%) | 7 | (9.5%) | 3 (4.1%) | 6 (8.3%) |
| stop of study medication | | | | | |
| Any TEAE leading to a temporary | 0 | 2 | (2.7%) | 2 (2.7%) | 1 (1.4%) |
| stop of study medication | | | | | |

TEAE = treatment-emergent adverse event

During the treatment period and follow-up, individual adverse events (AEs) were reported in less than 10% of subjects within a treatment group, except for diarrhea and nausea. Diarrhea was reported in 6.9% (5/72), 6.8% (5/74), 27.0% (20/74), and 16.7% (12/72) of subjects in the placebo, Compound (1) 300 mg bid, Compound (1) 600 mg bid, and Compound (1) 600 mg bid+oseltamivir 75 mg bid treatment groups, respectively. Nausea was reported in 0%, 4.1% (3/74), 4.1% (3/74), and 11.1% (8/72) of subjects in the placebo, Compound (1) 300 mg bid, Compound (1) 600 mg bid, and Compound (1) 600 mg bid+oseltamivir 75 mg bid treatment groups, respectively (Table 20).

TABLE 20
Number (%) of Subjects with Treatment-emergent Adverse Events; Safety Set.

| MedDRA System Organ | Cmpd (1) | | Cmpd (1) | | Cmpd (1) | Cmpd (1) | |
|---|---|---|---|---|---|---|---|
| Class | placebo | | 300 mg | | 600 mg | 600 mg | |
| Dictionary-derived Term | OST placebo | | OST placebo | | OST placebo | OST 75 mg | |
| Safety Set | 72 | | 74 | | 74 | 72 | |
| All events, n (%) | 32 | (44.4%) | 32 | (43.2%) | 39 (52.7%) | 29 | (40.3%) |
| Gastrointestinal disorders | 7 | (9.7%) | 10 | (13.5%) | 22 (29.7%) | 18 | (25.0%) |
| Diarrhoea | 5 | (6.9%) | 5 | (6.8%) | 20 (27.0%) | 12 | (16.7%) |
| Nausea | 0 | | 3 | (4.1%) | 3 (4.1%) | 8 | (11.1%) |
| Vomiting | 0 | | 1 | (1.4%) | 3 (4.1%) | 6 | (8.3%) |
| Abdominal pain upper | 0 | | 0 | | 2 (2.7%) | 1 | (1.4%) |
| Dry mouth | 0 | | 0 | | 1 (1.4%) | 1 | (1.4%) |
| Abdominal pain | 1 | (1.4%) | 0 | | 0 | 1 | (1.4%) |
| Abdominal discomfort | 0 | | 0 | | 1 (1.4%) | 0 | |
| Abdominal tenderness | 0 | | 0 | | 0 | 1 | (1.4%) |
| Aphthous stomatitis | 0 | | 0 | | 0 | 1 | (1.4%) |
| Constipation | 0 | | 0 | | 1 (1.4%) | 0 | |
| Dyspepsia | 0 | | 1 | (1.4%) | 0 | 0 | |
| Enteritis | 1 | (1.4%) | 0 | | 0 | 0 | |
| Tongue discolouration | 0 | | 1 | (1.4%) | 0 | 0 | |
| Investigations | 12 | (16.7%) | 8 | (10.8%) | 10 (13.5%) | 8 | (11.1%) |
| Blood creatine | 5 | (6.9%) | 2 | (2.7%) | 1 (1.4%) | 3 | (4.2%) |
| phosphokinase increased | | | | | | | |
| Glomerular filtration rate | 2 | (2.8%) | 4 | (5.4%) | 1 (1.4%) | 1 | (1.4%) |
| decreased | | | | | | | |
| Neutrophil count decreased | 0 | | 1 | (1.4%) | 4 (5.4%) | 1 | (1.4%) |
| Alanine aminotransferase | 0 | | 1 | (1.4%) | 2 (2.7%) | 1 | (1.4%) |
| increased | | | | | | | |
| Blood glucose increased | 0 | | 1 | (1.4%) | 1 (1.4%) | 2 | (2.8%) |
| Aspartate aminotransferase | 0 | | 1 | (1.4%) | 1 (1.4%) | 1 | (1.4%) |
| increased | | | | | | | |
| Gamma- | 1 | (1.4%) | 0 | | 1 (1.4%) | 0 | |
| glutamyltransferase | | | | | | | |
| increased | | | | | | | |
| Haemoglobin decreased | 0 | | 2 | (2.7%) | 0 | 0 | |
| White blood cell count | 0 | | 0 | | 1 (1.4%) | 1 | (1.4%) |
| decreased | | | | | | | |
| Blood bicarbonate | 1 | (1.4%) | 0 | | 0 | 0 | |
| decreased | | | | | | | |
| Blood bilirubin decreased | 1 | (1.4%) | 0 | | 0 | 0 | |
| Blood bilirubin increased | 0 | | 1 | (1.4%) | 0 | 0 | |
| Blood creatinine increased | 0 | | 1 | (1.4%) | 0 | 0 | |
| Blood potassium increased | 0 | | 0 | | 1 (1.4%) | 0 | |
| Blood pressure diastolic | 0 | | 0 | | 1 (1.4%) | 0 | |
| increased | | | | | | | |
| Blood urine | 0 | | 1 | (1.4%) | 0 | 0 | |
| Electrocardiogram ST | 0 | | 0 | | 0 | 1 | (1.4%) |
| segment depression | | | | | | | |
| HIV antibody positive | 1 | (1.4%) | 0 | | 0 | 0 | |
| HIV test positive | 1 | (1.4%) | 0 | | 0 | 0 | |
| Haemoglobin increased | 1 | (1.4%) | 0 | | 0 | 0 | |
| Hepatic enzyme increased | 0 | | 0 | | 1 (1.4%) | 0 | |
| Percussion test abnormal | 0 | | 1 | (1.4%) | 0 | 0 | |
| Protein urine | 0 | | 1 | (1.4%) | 0 | 0 | |
| Protein urine present | 0 | | 0 | | 0 | 1 | (1.4%) |
| Red blood cells urine | 0 | | 1 | (1.4%) | 0 | 0 | |
| Urine ketone body present | 0 | | 1 | (1.4%) | 0 | 0 | |
| Infections and infestations | 9 | (12.5%) | 5 | (6.8%) | 1 (1.4%) | 3 | (4.2%) |
| Bronchitis | 4 | (5.6%) | 1 | (1.4%) | 0 | 0 | |
| Sinusitis | 0 | | 3 | (4.1%) | 0 | 0 | |
| Oral herpes | 0 | | 0 | | 1 (1.4%) | 1 | (1.4%) |
| Bacterial vaginosis | 1 | (1.4%) | 0 | | 0 | 0 | |
| Fungal infection | 0 | | 0 | | 0 | 1 | (1.4%) |
| Hepatitis C | 0 | | 1 | (1.4%) | 0 | 0 | |
| Herpes simplex | 1 | (1.4%) | 0 | | 0 | 0 | |
| Labyrinthitis | 0 | | 0 | | 0 | 1 | (1.4%) |
| Otitis media | 1 | (1.4%) | 0 | | 0 | 0 | |
| Pharyngitis streptococcal | 1 | (1.4%) | 0 | | 0 | 0 | |
| Pneumonia chlamydial | 1 | (1.4%) | 0 | | 0 | 0 | |
| Upper respiratory tract | 1 | (1.4%) | 0 | | 0 | 0 | |
| infection | | | | | | | |
| Urinary tract infection | 1 | (1.4%) | 0 | | 0 | 0 | |
| Metabolism and nutrition | 4 | (5.6%) | 4 | (5.4%) | 5 (6.8%) | 4 | (5.6%) |
| disorders | | | | | | | |
| Hyperglycaemia | 0 | | 0 | | 2 (2.7%) | 1 | (1.4%) |
| Hyperkalaemia | 1 | (1.4%) | 0 | | 2 (2.7%) | 0 | |
| Hypokalaemia | 2 | (2.8%) | 0 | | 0 | 1 | (1.4%) |
| Hypophosphataemia | 0 | | 2 | (2.7%) | 0 | 0 | |
| Decreased appetite | 0 | | 0 | | 0 | 1 | (1.4%) |
| Dehydration | 1 | (1.4%) | 0 | | 0 | 0 | |
| Diabetes mellitus | 0 | | 1 | (1.4%) | 0 | 0 | |
| Gout | 0 | | 1 | (1.4%) | 0 | 0 | |
| Hyperproteinaemia | 0 | | 1 | (1.4%) | 0 | 0 | |
| Hyperuricaemia | 0 | | 0 | | 0 | 1 | (1.4%) |
| Hyponatraemia | 0 | | 1 | (1.4%) | 0 | 0 | |
| Increased appetite | 0 | | 0 | | 1 (1.4%) | 0 | |
| Nervous system disorders | 2 | (2.8%) | 4 | (5.4%) | 4 (5.4%) | 3 | (4.2%) |
| Headache | 0 | | 2 | (2.7%) | 1 (1.4%) | 2 | (2.8%) |
| Dizziness | 0 | | 0 | | 2 (2.7%) | 1 | (1.4%) |
| Balance disorder | 1 | (1.4%) | 0 | | 0 | 0 | |
| Dysgeusia | 0 | | 0 | | 0 | 1 | (1.4%) |
| Migraine | 0 | | 0 | | 1 (1.4%) | 0 | |
| Paraesthesia | 0 | | 1 | (1.4%) | 0 | 0 | |
| Syncope | 0 | | 1 | (1.4%) | 0 | 0 | |
| Tremor | 1 | (1.4%) | 0 | | 0 | 0 | |
| Renal and urinary disorders | 3 | (4.2%) | 6 | (8.1%) | 1 (1.4%) | 2 | (2.8%) |
| Proteinuria | 1 | (1.4%) | 3 | (4.1%) | 0 | 1 | (1.4%) |
| Renal impairment | 0 | | 1 | (1.4%) | 0 | 1 | (1.4%) |
| Dysuria | 1 | (1.4%) | 0 | | 0 | 0 | |
| Haematuria | 0 | | 1 | (1.4%) | 0 | 0 | |
| Micturition urgency | 0 | | 0 | | 1 (1.4%) | 0 | |
| Nephropathy | 1 | (1.4%) | 0 | | 0 | 0 | |
| Renal failure | 0 | | 1 | (1.4%) | 0 | 0 | |
| Renal failure chronic | 0 | | 1 | (1.4%) | 0 | 0 | |
| Blood and lymphatic system | 3 | (4.2%) | 3 | (4.1%) | 3 (4.1%) | 3 | (4.2%) |
| disorders | | | | | | | |
| Neutropenia | 1 | (1.4%) | 0 | | 1 (1.4%) | 1 | (1.4%) |
| Thrombocytopenia | 1 | (1.4%) | 1 | (1.4%) | 0 | 1 | (1.4%) |
| Lymphadenopathy | 1 | (1.4%) | 1 | (1.4%) | 0 | 0 | |
| Anaemia | 0 | | 0 | | 0 | 1 | (1.4%) |
| Leukopenia | 0 | | 0 | | 1 (1.4%) | 0 | |
| Lymph node pain | 0 | | 1 | (1.4%) | 0 | 0 | |
| Lymphopenia | 0 | | 0 | | 1 (1.4%) | 0 | |
| Respiratory, thoracic and | 2 | (2.8%) | 1 | (1.4%) | 2 (2.7%) | 1 | (1.4%) |
| mediastinal disorders | | | | | | | |
| Cough | 0 | | 0 | | 1 (1.4%) | 1 | (1.4%) |
| Wheezing | 0 | | 1 | (1.4%) | 1 (1.4%) | 0 | |
| Dyspnoea | 0 | | 1 | (1.4%) | 0 | 0 | |
| Nasal congestion | 1 | (1.4%) | 0 | | 0 | 0 | |
| Rhonchi | 0 | | 1 | (1.4%) | 0 | 0 | |
| Sinus congestion | 1 | (1.4%) | 0 | | 0 | 0 | |
| Cardiac disorders | 2 | (2.8%) | 1 | (1.4%) | 1 (1.4%) | 2 | (2.8%) |
| Atrioventricular block first | 1 | (1.4%) | 0 | | 0 | 0 | |
| degree | | | | | | | |
| Bradycardia | 0 | | 0 | | 1 (1.4%) | 0 | |
| Myocarditis | 1 | (1.4%) | 0 | | 0 | 0 | |
| Sinus bradycardia | 0 | | 0 | | 0 | 1 | (1.4%) |
| Sinus tachycardia | 0 | | 1 | (1.4%) | 0 | 0 | |
| Tachycardia | 0 | | 0 | | 0 | 1 | (1.4%) |
| Psychiatric disorders | 0 | | 3 | (4.1%) | 3 (4.1%) | 0 | |
| Insomnia | 0 | | 2 | (2.7%) | 1 (1.4%) | 0 | |
| Irritability | 0 | | 0 | | 1 (1.4%) | 0 | |
| Sleep disorder | 0 | | 0 | | 1 (1.4%) | 0 | |
| Sleep terror | 0 | | 1 | (1.4%) | 0 | 0 | |
| Skin and subcutaneous tissue | 2 | (2.8%) | 0 | | 2 (2.7%) | 2 | (2.8%) |
| disorders | | | | | | | |
| Dry skin | 0 | | 0 | | 1 (1.4%) | 0 | |
| Ecchymosis | 1 | (1.4%) | 0 | | 0 | 0 | |
| Hyperhidrosis | 0 | | 0 | | 1 (1.4%) | 0 | |
| Petechiae | 0 | | 0 | | 0 | 1 | (1.4%) |
| Pruritus | 0 | | 0 | | 0 | 1 | (1.4%) |
| Rash | 1 | (1.4%) | 0 | | 0 | 0 | |
| General disorders and | 1 | (1.4%) | 2 | (2.7%) | 0 | 2 | (2.8%) |
| administration site | | | | | | | |
| conditions | | | | | | | |
| Fatigue | 0 | | 1 | (1.4%) | 0 | 0 | |
| Hypothermia | 0 | | 1 | (1.4%) | 0 | 0 | |
| Influenza like illness | 1 | (1.4%) | 0 | | 0 | 0 | |
| Oedema peripheral | 0 | | 0 | | 0 | 1 | (1.4%) |
| Sensation of foreign body | 0 | | 0 | | 0 | 1 | (1.4%) |
| Musculoskeletal and | 1 | (1.4%) | 2 | (2.7%) | 1 (1.4%) | 1 | (1.4%) |
| connective tissue disorders | | | | | | | |

| | | | | | |
|---|---|---|---|---|---|
| Back pain | 0 | 1 | (1.4%) | 1 (1.4%) | 0 |

| | | | | | |
|---|---|---|---|---|---|
| Muscle spasms | 1 | (1.4%) | 0 | 0 | 0 |

| | | | | | |
|---|---|---|---|---|---|
| Musculoskeletal chest pain | 0 | 1 | (1.4%) | 0 | 0 |

| Myalgia | 0 | | 0 | 0 | 1 | (1.4%) |
|---|---|---|---|---|---|---|
| Ear and labyrinth disorders | 1 | (1.4%) | 0 | 0 | 3 | (4.2%) |

| | | | | | |
|---|---|---|---|---|---|
| Ear discomfort | 1 | (1.4%) | 0 | 0 | 0 |

| Middle ear effusion | 0 | 0 | 0 | 1 | (1.4%) |
|---|---|---|---|---|---|
| Tinnitus | 0 | 0 | 0 | 1 | (1.4%) |
| Vertigo | 0 | 0 | 0 | 1 | (1.4%) |
| Injury, poisoning and | 0 | 0 | 1 (1.4%) | 0 | |
| procedural complications | | | | | |
| Contusion | 0 | 0 | 1 (1.4%) | 0 | |
| Vascular disorders | 0 | 0 | 0 | 1 | (1.4%) |
| Hypertension | 0 | 0 | 0 | 1 | (1.4%) |

n = number of subjects with 1 or more events;
OST = oseltamivir

Other Adverse Events of Interest

No deaths were reported, however, two serious AEs (SAES) have been reported. For one subject in the Compound (1) 600 mg bid treatment group a moderate treatment-emergent AE (TEAE) of increased alanine aminotransferase was reported. This SAE started on Day 14 and was reported resolved 21 weeks later. The event was considered doubtfully related to study drug by the investigator. For another subject in the placebo treatment group, a severe SAE of thrombocytopenia was reported and considered possibly related to study drug by the investigator. The SAE started on Day 63 and was resolved 5 weeks later.

A summary of subjects with severe TEAEs is provided in Table 19. No life threatening TEAEs were reported.

TABLE 21
Number (%) of Subjects with Treatment-Emergent
Grade 3 Adverse Events; Safety Set.

| MedDRA System Organ | Cmpd (1) | Cmpd (1) | Cmpd (1) | Cmpd (1) |
|---|---|---|---|---|
| Class | placebo + | 300 mg + | 600 mg + | 600 mg + |
| Dictionary-derived Term | OST placebo | OST placebo | OST placebo | OST 75 mg |
| Safety Set | 72 | 74 | 74 | 72 |
| Any Severe (Grade 3) | 4 (5.6%) | 2 (2.7%) | 5 (6.8%) | 3 (4.2%) |
| TEAE | | | | |
| Blood and lymphatic | 2 (2.8%) | 0 | 1 (1.4%) | 1 (1.4%) |
| system disorders | | | | |
| Anaemia | 0 | 0 | 0 | 1 (1.4%) |
| Lymphopenia | 0 | 0 | 1 (1.4%) | 0 |
| Neutropenia | 1 (1.4%) | 0 | 0 | 0 |
| Thrombocytopenia | 1 (1.4%) | 0 | 0 | 0 |
| Gastrointestinal disorders | 0 | 0 | 2 (2.7%) | 1 (1.4%) |
| Constipation | 0 | 0 | 1 (1.4%) | 0 |
| Diarrhoea | 0 | 0 | 1 (1.4%) | 0 |
| Nausea | 0 | 0 | 0 | 1 (1.4%) |
| Vomiting | 0 | 0 | 0 | 1 (1.4%) |
| Investigations | 0 | 0 | 1 (1.4%) | 2 (2.8%) |
| Blood creatine | 0 | 0 | 1 (1.4%) | 0 |
| phosphokinase increased | | | | |
| Neutrophil count | 0 | 0 | 0 | 1 (1.4%) |
| decreased | | | | |
| Protein urine present | 0 | 0 | 0 | 1 (1.4%) |
| Metabolism and nutrition | 1 (1.4%) | 0 | 1 (1.4%) | 0 |
| disorders | | | | |
| Hyperkalaemia | 1 (1.4%) | 0 | 1 (1.4%) | 0 |
| Nervous system disorders | 1 (1.4%) | 1 (1.4%) | 0 | 0 |
| Balance disorder | 1 (1.4%) | 0 | 0 | 0 |
| Syncope | 0 | 1 (1.4%) | 0 | 0 |
| General disorders and | 0 | 1 (1.4%) | 0 | 0 |
| administration site | | | | |
| conditions | | | | |
| Fatigue | 0 | 1 (1.4%) | 0 | 0 |
| Infections and | 0 | 1 (1.4%) | 0 | 0 |
| infestations | | | | |
| Bronchitis | 0 | 1 (1.4%) | 0 | 0 |

TEAE = treatment-emergent adverse event

All but one severe TEAE were reported during treatment. Thrombocytopenia was reported after end of treatment in one subject in the placebo group; this TEAE was serious and is described above.

Eight other severe events were considered possibly related to trial medication by the investigator: neutropenia and balance disorder (placebo group); blood creatine phosphokinase increased and diarrhea (Compound (1) 600 mg bid group); and nausea, vomiting, protein urine present, neutrophil count decreased (Compound (1) 600 mg bid+oseltamivir 75 mg bid). The other severe TEAEs were considered not related or doubtfully related to trial medication.

Laboratory Findings

A summary of treatment-emergent worst laboratory toxicities of grade 3 or 4 is given in Table 22.

TABLE 22
Tabulation of the worst treatment-emergent toxicity grade 3 or 4; safety set.

| | Cmpd (1) | Cmpd (1) | Cmpd (1) | | Cmpd (1) |
|---|---|---|---|---|---|
| | placebo | 300 mg | 600 mg | | 600 mg |
| | OST placebo | OST placebo | OST placebo | | OST 75 mg |
| Analysis Set: | 72 | 74 | 74 | | 72 |
| Safety Set | | | | | |
| Chemistry | | | | | |
| CPK | | | | | |
| Grade 3 | 2 (2.8%) | 1 (1.4%) | 2 | (2.9%) | 2 (2.8%) |
| Grade 4 | 0 | 0 | 1 | (1.4%) | 0 |
| Cholesterol | | | | | |
| Grade 3 | 2 (2.8%) | 0 | 4 | (5.7%) | 7 (9.9%) |
| Glucose | | | | | |
| hyperglycemia | | | | | |
| N | 71 | 73 | 70 | | 71 |
| Grade 3 | 0 | 1 (1.4%) | 0 | | 0 |
| Hyperkalemia | | | | | |
| Grade 3 | 2 (2.8%) | 1 (1.4%) | 1 | (1.4%) | 1 (1.4%) |
| Grade 4 | 0 | 0 | 2 | (2.9%) | 0 |
| Hypernatremia | | | | | |
| Grade 3 | 0 | 0 | 0 | | 1 (1.4%) |
| Grade 4 | 0 | 0 | 0 | | 1 (1.4%) |
| Hypocalcemia | | | | | |
| Grade 4 | 0 | 1 (1.4%) | 0 | | 0 |
| Hypoglycemia | | | | | |
| Grade 3 | 0 | 0 | 2 | (2.9%) | 0 |
| Hypokalemia | | | | | |
| Grade 3 | 1 (1.4%) | 0 | 1 | (1.4%) | 0 |
| Grade 4 | 1 (1.4%) | 0 | 1 | (1.4%) | 1 (1.4%) |
| Hypomagnesemia | | | | | |
| Grade 3 | 0 | 1 (1.4%) | 0 | | 0 |
| Hyponatremia | | | | | |
| Grade 3 | 1 (1.4%) | 0 | 0 | | 1 (1.4%) |
| Hypophosphatemia | | | | | |
| Grade 3 | 2 (2.8%) | 2 (2.7%) | 0 | | 1 (1.4%) |
| ALT increase by | | | | | |
| factor | | | | | |
| Grade 3 | 0 | 0 | 1 | (1.4%) | 1 (1.4%) |
| Grade 4 | 0 | 0 | 1 | (1.4%) | 0 |
| AST increase by | | | | | |
| factor | | | | | |
| Grade 4 | 0 | 0 | 1 | (1.4%) | 0 |
| Hematology | | | | | |
| Hemoglobin - change | | | | | |
| from baseline | | | | | |
| Grade 3 | 1 (1.5%) | 5 (7.1%) | 3 | (4.4%) | 6 (8.7%) |
| Neutrophils decrease | | | | | |
| Grade 3 | 1 (1.4%) | 1 (1.4%) | 4 | (5.7%) | 1 (1.4%) |
| Grade 4 | 0 | 0 | 0 | | 1 (1.4%) |
| Urinalysis | | | | | |
| Urine RBC increased | | | | | |
| Grade 3 | 0 | 3 (9.4%) | 5 | (15.6%) | 3 (8.1%) |
| Urine WBC increased | | | | | |
| Grade 3 | 0 | 0 | 2 | (4.7%) | 2 (4.3%) |
| Urine glucose | | | | | |
| increased | | | | | |
| Grade 3 | 1 (1.4%) | 1 (1.4%) | 0 | | 1 (1.4%) |
| Urine protein | | | | | |
| increased | | | | | |
| Grade 3 | 0 | 0 | 2 | (2.9%) | 3 (4.3%) |

ALT = alanine aminotransferase,
AST = aspartate aminotransferase;
CPK = creatine phosphokinase;
RBC = red blood cell;
WBC = white blood cell

Apparent differences between the active treatment groups and placebo were seen in grade 3 cholesterol increases, hemoglobin changes from baseline, urine erythrocytes increases, and urine protein increases, however, no clinically relevant differences in related TEAEs were observed.

Conclusions

Primary Efficacy

Treatment with Compound (1) resulted in a statistically significant and dose-dependent decrease in AUC of viral load (by qRT-PCR) over 7 days from start of dosing. Further, Compound (1) in combination with oseltamivir resulted in a statistically significant lower AUC of viral load (by qRT-PCR) as compared to Compound (1) alone (600-mg dose).

Secondary Efficacy

Little separation in time to resolution of 7 primary influenza symptoms was found by the patient-reported outcome assessment, Flu-iiQ. Given that the trial was finalized at the interim analysis for early success, the sample sizes per arm were relatively small, and clinical outcome comparisons had low power to show differences. The viral culture data were confirmatory of the qRT-PCR results but showed a shorter time to negativity compared to the qRT-PCR data.

Safety

Compound (1) was generally safe and well tolerated. A favorable safety profile was established. Increased incidences of diarrhea were reported; more common with 600 mg Compound (1) (as mono- or combination therapy). No safety concerns were noted regarding laboratory values, electrocardiograms, and vital signs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating or reducing the severity of influenza virus infection comprising administering to a patient infected with influenza from about 200 mg to about 800 mg twice per day Compound (1) or a pharmaceutically acceptable salt thereof, wherein Compound (1) has the structure:

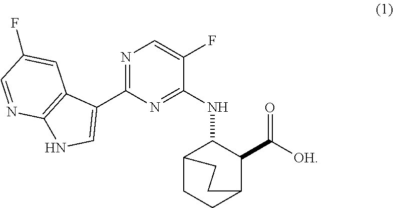 [SMILES: O=C(O)[C@H]1C2CCC(CC2)[C@@H]1Nc1nc(-c2c[nH]c3ncc(F)cc23)ncc1F]

2. The method of claim 1, wherein the patient is administered a crystalline form of the HCl salt of Compound (1).

3. The method of claim 2, wherein the patient is administered from about 250 mg to about 750 mg of Compound (1).

4. The method of claim 1, wherein Compound (1) or a pharmaceutically acceptable salt thereof is administered to the patient every day for 3 to 10 days.

5. The method of claim 1, wherein the influenza virus is influenza A virus.

6. The method of claim 1, comprising administering about 600 mg of Compound (1) twice per day.

7. The method of any one of claims 1-6, further comprising administering an additional therapeutic agent.

8. The method of claim 7, wherein the additional therapeutic agent is a neuraminidase inhibitor.

9. The method of claim 8, wherein the neuraminidase inhibitor is oseltamivir or a pharmaceutically acceptable salt thereof.

10. The method of claim 9, further comprising administering from about 50 mg to about 100 mg of oseltamivir at least once per day.

11. The method of claim 9, comprising administering about 75 mg of oseltamivir at least once per day.

12. The method of claim 9, comprising co-administering about 75 mg of oseltamivir twice per day with said Compound (1) or pharmaceutically acceptable salt thereof.

13. A method of treating or reducing the severity of influenza virus infection comprising administering to a patient infected with influenza a pharmaceutical combination comprising from about 200 mg to about 800 mg of Compound (1) or a pharmaceutically acceptable salt thereof, and from about 50 mg to about 100 mg of oseltamivir or a pharmaceutically acceptable salt thereof at least once per day, wherein Compound (1) has the structure: 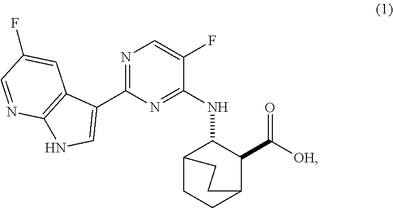 [SMILES: O=C(O)[C@H]1C2CCC(CC2)[C@@H]1Nc1nc(-c2c[nH]c3ncc(F)cc23)ncc1F]
and
wherein said administration is first effected within 48 to 96 hours of onset of at least one influenza symptom in said patient.

14. The method of claim 13, wherein said administration is first effected within about 60 to about 96 hours of said onset of influenza symptom in said patient.

15. The method of either of claim 13 or 14, wherein said administration is first effected within about 72 to about 96 hours of said onset of influenza symptom in said patient.

16. The method of any one of claims 13-15 wherein the influenza symptom includes at least one symptom selected from nasal congestion, sore throat, cough, aches, fatigue, headaches, and chills/sweats.

17. The method of any one of claims 13-16, wherein the combination comprises from about 300 mg to about 600 mg of Compound (1) or a pharmaceutically acceptable salt thereof.

18. The method of any one of claims 13-17, wherein the combination comprises about 600 mg of Compound (1) or a pharmaceutically acceptable salt thereof.

19. The method of any one of claims 13-18, wherein the combination comprises about 75 mg of a neuraminidase inhibitor.

20. The method of claim 19, wherein the neuraminidase inhibitor is oseltamivir or a pharmaceutically acceptable salt thereof.

21. The method of any one of claims 13-20, wherein the combination comprises about 600 mg of Compound (1) or a pharmaceutically acceptable salt thereof and about 75 mg of oseltamivir or a pharmaceutically acceptable salt thereof.

22. The method of any one of claims 13-21, wherein the combination comprises about 600 mg of Compound (1) or a pharmaceutically acceptable salt thereof and about 75 mg of oseltamivir or a pharmaceutically acceptable salt thereof and the combination is administered twice per day.

23. The method of any one of claims 13-22, wherein the combination comprises about 600 mg of Compound (1) or a pharmaceutically acceptable salt thereof and about 75 mg of oseltamivir or a pharmaceutically acceptable salt thereof, the combination is administered twice per day, and said administration is first effected within about 72 hours to about 96 hours of onset of symptoms of said influenza.

24. The method of any one of claims 13-23, wherein the combination comprises a crystalline form of the HCl salt of Compound (1).

25. The method of any one of claims 13-24, wherein the oseltamivir or a pharmaceutically acceptable salt thereof is oseltamivir phosphate.

26. The method of any one of claims 13-25, wherein the combination is administered to the patient every day for 3 to 10 days.

27. The method of any one of claims 13-26, wherein the influenza virus is influenza A virus.

28. The method of claim 13, wherein said administration is first effected after the patient's oxygen saturation level has fallen below 94%, as measured by pulse oximetry or after the patient has been administered supplemental oxygen.

29. A kit for treating or reducing the severity of influenza virus infection comprising Compound (1) or a pharmaceutically acceptable salt thereof or a pharmaceutical composition comprising compound (1) or a pharmaceutically acceptable salt thereof and at least one leaflet comprising prescribing information, wherein said prescribing information comprises the method as described in any of claims 1 to 28 and Compound (1) has the structure:

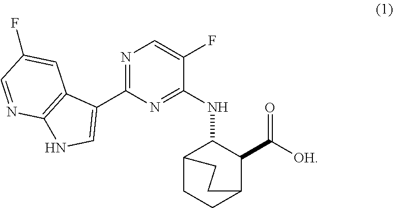 [SMILES: O=C(O)[C@H]1C2CCC(CC2)[C@@H]1Nc1nc(-c2c[nH]c3ncc(F)cc23)ncc1F]

30. The kit of claim 29, wherein the kit comprises a crystalline form of the HCl salt of Compound (1).
